(19) United States
(12) Patent Application Publication
(10) Pub. No.: US 2014/0206002 A1
(43) Pub. Date: Jul. 24, 2014

(54) Methods of Diagnosing Breast Cancer

(71) Applicant: THE TRUSTEES OF THE UNIVERSITY OF PENNSYLVANIA, Philadelphia, PA (US)

(72) Inventors: Roger A. Greenberg, Bala Cynwyd, PA (US); Robert Winqvist, Oulu (FI); Szilvia Solyom, Baltimore, MD (US); Katri Pylkas, Oulu (FI)

(21) Appl. No.: 14/157,698

(22) Filed: Jan. 17, 2014

Related U.S. Application Data
(60) Provisional application No. 61/753,724, filed on Jan. 17, 2013.

(51) Int. Cl. C12Q 1/68

(52) U.S. Cl. CPC C12Q 1/6886
USPC 435/6.11; 536/23.5

(57) ABSTRACT

The disclosed subject matter is based on the discovery that a mutation (a single nucleotide polymorphism or “SNP”) in the gene encoding Abraxas (also referred to as ABRA1, CCDC98 or FAM175A), is associated with susceptibility to cancer, e.g., breast cancer. In particular, the disclosed subject matter is based on the identification of a heterozygous alteration in the Abraxas gene that is associated with breast cancer and specifically correlated with familial cancer. Accordingly, the SNP disclosed herein is useful for diagnosing, prognosing, screening for, and evaluating predisposition to cancer in humans. The disclosed subject matter also provides nucleic acid molecules containing the SNP, methods and reagents for the detection of the SNP, and assays or kits for detection of the SNP.

This application claims priority to U.S. Provisional App. Ser. No. 61/753,724, filed Jan. 17, 2013, the disclosure of which is hereby incorporated by reference in its entirety.

### 1. GRANT INFORMATION

This invention was made with government support under 1R01CA138835-01 awarded by the National Cancer Institute. The government has certain rights in the invention.

### 2. BACKGROUND

Breast cancer is the most common malignancy affecting women. The presence of a family history is the most important single risk factor, with the first-degree relatives having approximately a two-fold increased risk over the general population. BRCA1 and BRCA2 are the two most important susceptibility genes in hereditary predisposition to breast and ovarian cancer. However, germline mutations in these tumor suppressors account for no more than 20% of the familial breast cancer cases worldwide (1). Most of the remaining cancer cases are predicted to involve mutations in moderate and low penetrance susceptibility genes together with influence from environmental factors, although the data from large multiple case families strongly suggest that there might still be additional high penetrance genes to be identified (2, 3). Improved knowledge on the functions of BRCA1 and BRCA2 in the DNA damage response pathway has been instrumental to the identification of a number of breast cancer susceptibility genes including PALB2, BRIP1, ATM and CHEK2, all with protein products interacting directly or indirectly with BRCA1 and BRCA2 (4, 5).

BRCA1 plays multifaceted roles as a gatekeeper of genomic integrity. It is essential for efficient DNA repair by homologous recombination and execution of cell cycle checkpoints activated by DNA damage (6). The carboxyl-terminal region of BRCA1 contains two BRCT (BRCA1 carboxyl-terminal) repeats that are critical for its tumor suppressor function. These BRCA1 BRCT motifs are the most common site of clinical missense mutations, which invariably disrupt phosphorylation dependent interactions with direct binding partners (7, 8).

Abraxas (also referred to as ABRA1, CCDC98 or FAM175A) serves as a central organizer of a large BRCA1 holoenzyme complex, directly binding via its phosphorylated C-terminus to the BRCA1 BRCT motifs (9-11). This interaction links BRCA1 to a core protein complex dedicated to ubiquitin chain recognition and hydrolysis at DNA double strand breaks (DSBs) (9, 12-16). Abraxas and the other members of this complex [RAP80, BRCC36, MERIT40/NBA1 (HUGO name BABAM1) and BRCC45] are required for DNA damage checkpoints and cellular resistance to ionizing radiation.

A deleterious germline RAP80 variant (del81E) that abrogates ubiquitin binding, DSB targeting and genome integrity has been reported in two breast cancer patients (17). Additionally, a genome-wide linkage consortium study suggested an association between the rare allele of SNP rs8170 in MERIT40/NBA1 and an increased propensity for hormone receptor-negative breast cancer, both in the general population and in BRCA1 mutation carriers (18). The same SNP has also been associated with susceptibility to serous epithelial ovarian cancer (19).

Identification of breast or ovarian cancer-associated mutations, in addition to known predictors such as BRCA1 and BRCA2, such as those involved in the BRCA1 core protein complex, are needed to more accurately diagnose the disease in a greater number of cases.

### 3. SUMMARY

The disclosed subject matter is based on the discovery that a mutation (a single nucleotide polymorphism or “SNP”) in the gene encoding Abraxas (also referred to as ABRA1, CCDC98 or FAM175A), is associated with susceptibility to cancer, e.g., breast cancer. In particular, the disclosed subject matter is based on the identification of a heterozygous alteration, c.1082G>A (Arg361Gln) in the Abraxas gene that is disease-associated and specifically correlated with familial cancer. Accordingly, the SNP disclosed herein is useful for diagnosing, prognosing, screening for, and evaluating predisposition to cancer in humans.

In one aspect, the disclosed subject matter provides a method of determining whether a subject has an increased risk for developing breast cancer, or has breast cancer, comprising determining, in a biological sample comprising a nucleic acid from the subject, the nucleotide present at nucleotide position 1082 of the Abraxas gene, where presence of an A at nucleotide position 1082 is indicative of an increased likelihood of the subject having breast cancer or developing breast cancer, as compared with a subject having a G at nucleotide position 1082. In one embodiment, the c.1082G>A polymorphism can be identified in conjunction with, or following, the determination of the presence or absence of other genetic mutations known to be associated with cancer, e.g., mutations in BRCA1 or BRCA2 or known mutations in other genes. The methods of the disclosed subject matter can also be used in conjunction with other known methods for diagnosing or predicting likelihood of developing cancer, e.g., breast cancer.

In one embodiment, the Abraxas gene has the nucleotide sequence of SEQ ID NO: 1, or the complement thereof. The nucleic acid can be a nucleic acid extract from a biological sample, e.g., blood, saliva, buccal cells, etc., obtained from the subject.

Determining the nucleotide present at position 1082 can be carried out by any method known in the art. In one embodiment, the nucleic acid amplification is carried out by polymerase chain reaction (PCR). In another embodiment, determining is performed using, for example, sequencing, 5′ nuclease digestion, molecular beacon assay, oligonucleotide ligation assay, size analysis, single-stranded conformation polymorphism analysis, or denaturing gradient gel electrophoresis (DGGE). In another embodiment, determining is performed using an allele-specific method. For example, the allele-specific method can be allele-specific probe hybridization, allele-specific primer extension, or allele-specific amplification. In one embodiment, the method for determining is an automated method.

In one embodiment, the subject is heterozygous for the A allele at nucleotide position 1082.

In another embodiment, the breast cancer is lobular breast cancer. In still another embodiment, the subject has a family history of cancer, and in particular a family history of breast cancer. In yet another embodiment, the subject has previously tested negative for a BRCA1 or BRCA2 cancer-associated mutation.

In another aspect, the disclosed subject matter provides a method of determining whether a subject has an increased risk for developing breast cancer, or has breast cancer, comprising determining, in a biological sample comprising Abraxas protein or relevant portion thereof from the subject, the amino acid present at amino acid position 361 of the Abraxas protein, where the presence of a glutamine at amino acid position 361 is indicative of an increased risk for developing breast cancer, or the presence of breast cancer, in the subject, as compared with a subject having an arginine at amino acid position 361. In one embodiment, the Abraxas protein has the amino acid sequence of SEQ ID NO: 2.

In yet another aspect, the disclosed subject matter provides a method for treating or preventing breast cancer in a subject comprising administering to the subject a therapeutic agent or a prophylactic agent or regime, the subject having been identified as having an increased risk for breast cancer based on the presence of an A at nucleotide position 1082 of the Abraxas gene. In one embodiment, the therapeutic or prophylactic agent includes surgery, chemotherapeutic agents, and/or radiation.

The disclosed subject matter also provides a nucleic acid molecule comprising all or a portion of the nucleic acid sequence of SEQ ID NO:1, where the nucleic acid molecule is at least 10 nucleotides in length and where the nucleic acid sequence includes a polymorphic site at nucleotide position 1082 of SEQ ID NO:1. In one embodiment, at the polymorphic site the nucleotide is different from that in a corresponding reference allele. In one embodiment, the nucleic acid is an allele-specific nucleic acid, e.g., a probe that hybridizes to the polymorphic site.

In another aspect, the disclosed subject matter provides a peptide comprising the amino acid sequence of SEQ ID NO:2 which is at least ten contiguous amino acids, where the peptide includes glutamine at amino acid position 361 of SEQ ID NO: 2.

In still another aspect, the disclosed subject matter provides kits for determining whether a subject has an increased risk for breast cancer, where the kit contains at least one container and at least one oligonucleotide stored in the container, where the oligonucleotide is capable of detecting the presence or absence of the polymorphism at position 1082 of the nucleotide sequence of SEQ ID NO:1, or its complement. In one embodiment, the oligonucleotide selectively hybridizes to the nucleic acid in the presence of the polymorphism and does not hybridize to the nucleic acid in the absence of the polymorphism.

### 4. BRIEF DESCRIPTIONS OF THE DRAWINGS

FIG. 1. Identification of Abraxas mutation c.1082G>A (Arg361Gln) in breast cancer index cases and cancer families. (A) Schematic diagram of the Abraxas protein and the site of Arg361Gln. Chromatogram of c.1082G>A (Arg361Gln) is displayed directly below the bipartite nuclear localization signal 358-Lys-Arg-Ser-Arg-361 and 368-Lys-Arg-Ser-Lys-371 shown as a hatched region. (B) Evolutionary conservation of Abraxas c.1082G and the encoded codon Arg361. The conservation scoring was performed by PRALINE. The scoring scheme extends from 0 for the least conserved alignment position, up to * for the most conserved alignment position. (C) Pedigrees of two Abraxas c.1082G>A mutation positive breast cancer families where segregation analysis was possible (BR-0194 and 96-653). Black circles represent breast (Br) cancer cases, other cancer types [brain, colon (Co), endometrial (End), ovarian (Ov), pancreatic (Pan), prostate (Pro), sarcoma (Sar), skin, stomach (Sto), Ca, unknown] are marked with grey circles (females) or squares (males). Arrows point to index patients. A slashed symbol indicates a deceased individual. Sample identification codes are provided for all individuals where DNA specimens were available for mutation status analysis. Individuals are marked with a plus sign if mutation-positive. The age at diagnosis is indicated below the patients. The age at monitoring (or age at death), when known, is shown for the healthy individuals.

FIG. 2. Abraxas R361Q disrupts nuclear localization and recruitment to DNA double-strand breaks (DSBs). (A) Immunofluorescence (IF) of HA-tagged Abraxas wild type (WT) and Abraxas R361Q transiently transfected in U2OS (top) and MCF10A (bottom) cells demonstrates cytoplasmic localization for the Abraxas mutant R361Q. DAPI, 4′,6-diamidino-2-phenylindole. (B) Quantification of cells presenting a predominantly nuclear staining in HeLa, U2OS and MCF10A stably expressing FLAG-HA Abraxas WT or R361Q. Error bars represent mean±SD. P values were calculated by an unpaired T-test. ***P<0.0001. (C) Immunoblot (IB) of complexes after FLAG immunoprecipitation (IP) of FLAG-HA-tagged Abraxas from whole-cell lysates of 293T cells (−) or 293T cells lines stably expressing either Abraxas WT or R361Q. The location of endogenous and ectopic Abraxas protein is indicated. (D) IB of FLAG-HA-tagged Abraxas complexes after FLAG-IP from cytoplasmic and nuclear fractions of HeLa S3 cell lines that maintain stable expression of either Abraxas WT or Abraxas R361Q.

FIG. 3. Abraxas R361Q impairs BRCA1 and RAP80 DNA damage response functions. (A) IF in U2OS cells at 7 hours after 10 Gy IR demonstrates decreased BRCA1 and RAP80 IRIF in cells expressing Abraxas R361Q. (B) Quantification of the percentage of HeLa, U2OS, and MCF10A cells expressing Abraxas WT or R361Q with greater than 5 BRCA1 or RAP80 foci per nucleus. The mean was calculated from more than 200 cells for each condition in triplicate. Error bars represent mean±SD. P values were calculated by an unpaired T-test. *P=0.0181; **P=0.0056; ***P<0.0001. (C) Clonogenic survival assay of U2OS cells stably expressing Abraxas WT or R361Q after exposure to the indicated doses of γ-radiation. Points represent the average of three independent experiments run in triplicate. Error bars represent mean±SD. (D) Evaluation of the IR induced G2-M checkpoint in transiently transfected 293T cells. Mitotic cells were detected by FACS using an antibody against phospho-histone H3. The mean mitotic cell population was calculated from three independent experiments in which, in each sample, 10,000 cells were examined. Error bars represent SD. P-values were calculated by the Student's T-test assuming unequal variances. ***P<0.0001. (E) Evaluation of the homology directed DNA repair of a nuclease-induced DSB after transfection of a DR-GFP reporter cell line with the indicated plasmids. GFP-positive cells indicate the presence of homology-directed DNA repair. Data are representative of four independent experiments. Error bars represent mean±SD. P-values were calculated by an unpaired T-test. **P=0.0028; ***P<0.0001.

FIG. 4. Abraxas R361Q is preferentially immunopreciptated with its interacting partners in the cytoplasm. (A) IB of FLAG-HA-tagged Abraxas complexes after FLAG-IP from cytoplasmic and nuclear fractions of HeLa S3 cell lines that maintain stable expression of either Abraxas wild-type or Abraxas R361Q. (B) IB of Abraxas showing endogenous (bottom band) and ectopic (top band) expression of the protein in nuclear lysates of HeLa S3 cell lines mock or that maintain stable expression of either Abraxas wild-type or Abraxas R361Q.

FIG. 5. Expression of Abraxas R361Q affects DNA damage response. (A) (Top) Schematic of the DSB reporter locus. The lac repressor (LacI) is fused to mCherry and to the nuclease domain of the FokI endonuclease, enabling DSB induction within the 9 kb of 256 lac operator repeats (29). (Bottom) Representative images of HA-tagged Abraxas wild-type and R361Q in U2OS DSB reporter cells. (B and C) Clonogenic survival assay of 293T (B) and MCF10A (C) cells stably expressing Abraxas wild-type or R361Q after exposure to the indicated doses of γ-radiation. Points represent the average of three independent experiments run in triplicate. Error bars represent mean±SD.

FIG. 6. Nuclear retention of Abraxas R361Q restores localization and recruitment to DSB. (A) U2OS cells stably expressing HA-tagged Abraxas wild-type or R361Q were treated with 20 nM Leptomycin B (+) or left untreated (−) for 2 hrs prior to 10 Gy IR. IF was performed 7 hrs after irradiation with an HA antibody. (B) U2OS cells stably expressing HA-tagged Abraxas wild-type or R361Q were treated with 20 nM Leptomycin B prior to 10 Gy IR. IF was performed 7 hrs after irradiation with BRCA1 or RAP80 antibodies. Representative images demonstrate that forced expression of the mutant Abraxas protein in the nucleus restores IRIF formation.

FIG. 7. Cytoplasmic localization of Abraxas R361Q does not aberrantly activate β-catenin. (A) Staining of U2OS cells stably expressing HA-tagged Abraxas wild-type or R361Q with a β-catenin antibody does not reveal any mislocalization of β-catenin. (B) Localization of β-catenin was assessed by western blotting after fractionation of HeLa S3 cells stably expressing Abraxas wild-type or R361Q. C: cytoplasm, N: Nucleus.

FIG. 8. Exemplary nucleic sequence of the Abraxas gene, represented by GenBank Accession No. EF531340 (SEQ ID NO:1).

FIG. 9. Exemplary amino acid sequence of the Abraxas protein, represented by GenBank Accession No. ABP87396.1 (SEQ ID NO:2).

### 5. DETAILED DESCRIPTION OF THE INVENTION

The present disclosed subject matter is based on the discovery that a mutation (a single nucleotide polymorphism or “SNP”) in the gene encoding Abraxas (also referred to as ABRA1, CCDC98 or FAM175A), is associated with susceptibility to cancer, e.g., breast cancer. The SNP disclosed herein is useful for diagnosing, prognosing, screening for, and evaluating predisposition to cancer in humans. Furthermore, the disclosed subject matter provides nucleic acid molecules containing the SNP, methods and reagents for the detection of the SNP, and assays or kits for detection of the SNP. Thus, the disclosed subject matter includes methods and compositions for identifying a subject at increased risk (as compared to a control subject) for developing cancer and/or the presence of cancer in a subject, by detecting the presence of a mutation associated with cancer in the gene encoding Abraxas and/or Abraxas isoforms (the altered protein products of a mutated Abraxas form), in a sample from the subject. The disclosed subject matter also provides methods and compositions for treating a subject by identifying the subject as having cancer or an increased risk for cancer, and subsequently administering treatment (or prophylactic) based on the identification of cancer or risk for cancer using the SNP described herein.

In one embodiment, the methods of the disclosed subject matter include determining the presence or absence of additional mutations associated with cancer in the Abraxas gene or in other genes, e.g., mutations in the BRCA1 and/or BRCA2 genes, as well as the presence or absence of the SNP described herein. The disclosed subject matter also provides methods and compositions for selecting or formulating a treatment regimen and predicting the likelihood of response of a subject to a particular treatment regimen, e.g., chemotherapy, of a patient with cancer, e.g., breast cancer.

As described herein, DNA from 125 Northern Finnish breast cancer families was studied for coding region and splice-site Abraxas mutations. A heterozygous alteration, c.1082G>A (Arg361Gln) in Abraxas was identified in three breast cancer families, and in one additional familial case from an unselected breast cancer cohort. This mutation was absent from 868 healthy female control individuals tested (see Table 3, below). The prevalence of c.1082G>A in familial cases as compared with control cases, and also in familial cases compared with unselected breast cancer cases, was found to be significantly different (P=0.002 and P=0.005, respectively), which indicates that this variant is disease-associated and specifically correlated with familial cancer. The c.1082G>A (Arg361Gln) mutation in Abraxas impairs nuclear localization of the protein, and disrupts BRCA1 DNA damage repair function, but does not affect the binding between Abraxas and BRCA.

It was found that of the Abraxas mutation-associated breast cancer cases, lobular histology was more common in the breast cancers than is typically seen. Furthermore, the four breast cancer cases for which data was available were all estrogen receptor-positive, progesterone receptor-positive, and HER2-negative. Thus, the Abraxas mutation identified herein can be used to predict increased likelihood of developing certain forms of breast cancer, e.g., lobular breast cancer. Lobular breast cancer may be more resistant to chemotherapy than other forms of breast cancer, and can be difficult to diagnose using breast examination.

In addition to breast cancer, Abraxas R361Q families displayed additional cancer types. Therefore, Abraxas R361Q may also be associated with cancers other than breast cancer and therefore can be used to predict increased likelihood of developing various cancers, including, for example, lung, lip, lymphoma and neuroblastoma.

Based on its exclusive occurrence in familial cancers, disease cosegregation, evolutionary conservation, and disruption of critical BRCA1 functions, the recurrent Abraxas c.1082G>A mutation is associated with cancer predisposition. The present disclosure identifies Abraxas as a new breast cancer susceptibility gene, and, without wishing to be being bound by theory, the disclosure supports the concept of a BRCA-centered tumor suppressor network.

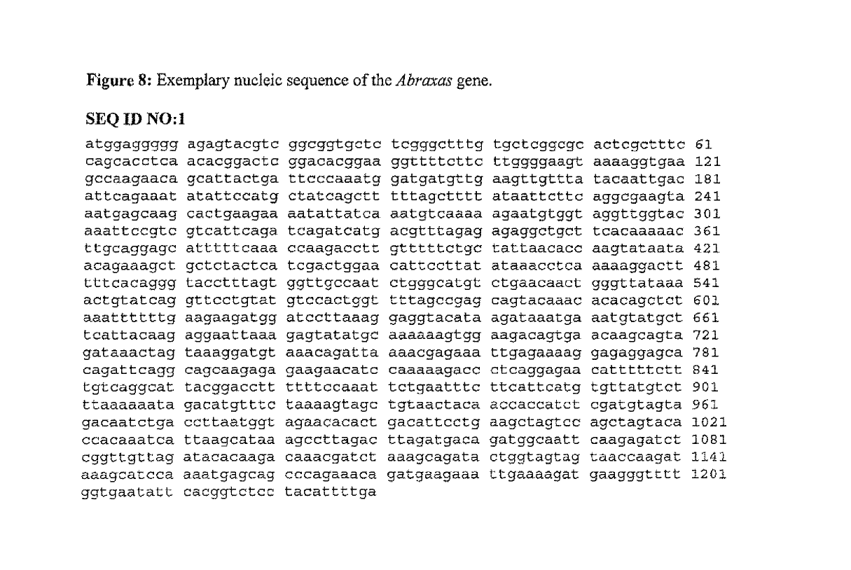

The Abraxas gene which includes the c.1082G>A SNP has at least two alleles, referred to herein as the reference allele and the variant allele. The reference allele (prototypical or wild type allele), which is the “0” allele, typically corresponds to the nucleotide sequence of the Abraxas gene which has been deposited with GenBank under Accession number EF531340 (mRNA sequence) (SEQ ID NO:1; FIG. 8). The variant allele (the “A” allele) differs from the reference allele at nucleotide residue 1082. The disclosed subject matter also relates to complements of the variant alleles.

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Throughout the specification, in identifying a SNP position, reference is generally made to the protein-encoding strand, only for the purpose of convenience.

The disclosed subject matter also relates to nucleic acid molecules which hybridize to and/or share identity with the variant alleles identified herein (or their complements) and which also include the variant nucleotide at the corresponding SNP site. Probes and primers can be designed to hybridize to either strand and SNP genotyping methods disclosed herein can generally target either strand.

The disclosed subject matter further relates to portions of the variant alleles and portions of complements of the variant alleles which include (encompass) the site of the SNP and are at least 5 nucleotides in length. Portions can be, for example, 5-10, 5-15, 10-20, 5-25, 10-30, 10-50 or 10-100 bases long. For example, a portion of a variant allele which is 21 nucleotides in length includes the single nucleotide polymorphism (the nucleotide which differs from the reference allele at that site) and twenty additional nucleotides which flank the site in the variant allele. These nucleotides can be on one or both sides of the single nucleotide polymorphism, e.g., the c.1082G>A polymorphism. Thus, the disclosed subject matter relates to a portion of the Abraxas gene having a nucleotide sequence as deposited in GenBank as Accession number EF531340 (SEQ ID NO:1; FIG. 8) comprising a single nucleotide polymorphism at nucleotide 1082. The nucleotide sequences of the disclosed subject matter can be double- or single-stranded.

The disclosed subject matter further provides a method of analyzing a nucleic acid from an individual to determine which base is present residue at 1082 of Abraxas or in the encoded protein at residue 361.

Accordingly, one aspect of the disclosed subject matter provides a method for diagnosing a genetic predisposition to cancer in a subject, comprising detecting, in a sample of biological material comprising at least one polynucleotide of a subject, a G>A point mutation at nucleotide 1082 of human Abraxas, where the presence of the variant allele (G) is associated with an increased susceptibility to developing cancer as compared to a subject having the wild type, or reference, allele (A).

In one embodiment, the methods of the disclosed subject matter include identifying the presence or absence of additional mutations associated with cancer in the same or other genes, e.g., mutations in the BRCA1 and/or BRCA2 genes. For example, a subject can be tested for the Abraxas polymorphism identified herein as part of a panel comprising other known cancer-associated polymorphisms. In another embodiment, a subject can be tested for the Abraxas polymorphism identified herein following testing for other known cancer-associated polymorphisms, e.g., where the subject tested negative for other cancer-associated polymorphisms. In one embodiment, the subject has a family history of cancer, e.g., breast cancer. Thus, the methods and compositions of the disclosed subject matter can be used in combination with other known predictive and diagnostic methods for identifying risk of cancer in a subject, e.g., in combination with identification of mutations in BRCA1 and/or BRCA2, or other predictive genes, family history, physical examination, mammography, etc.

Yet another aspect of the disclosed subject matter provides a method for treating cancer comprising detecting, in a sample of biological material comprising at least one polynucleotide from a subject, a G>A point mutation at nucleotide 1082 of human Abraxas, and treating the subject in such a way as to treat cancer, e.g., breast cancer, in the subject.

Still another aspect of the disclosed subject matter provides a method for the prophylactic treatment of a subject identified with a genetic predisposition to cancer identified through the measurement of the c.1082G>A polymorphism in Abraxas.

In another embodiment, methods and compositions of the disclosed subject matter are used to identify subjects who are likely to be responsive or unresponsive to treatment, e.g., chemotherapy, depending on the presence or absence of the identified Abraxas mutation. In one embodiment, the c.1082G>A polymorphic site in the Abraxas gene can be used to predict the likelihood of a subject developing lobular breast cancer. Lobular breast cancer may be more resistant to chemotherapy than other forms of breast cancer. Therefore, a determination that a subject has or is at risk for lobular breast cancer can be used in to determine a treatment regime for the subject.

Another aspect of the disclosed subject matter provides an isolated nucleic acid sequence comprising at least about 5, 10, 15, 20, 25 or 30 contiguous nucleotides or their complements found in the genomic sequences adjacent to and including the c.1082G>A polymorphic site in the Abraxas gene.

### DEFINITIONS

A nucleic acid molecule or oligonucleotide can be DNA or RNA, and single- or double-stranded. Nucleic acid molecules and oligonucleotides can be naturally occurring or non-naturally occurring (e.g., synthetic), but are typically prepared by synthetic means. Preferred nucleic acid molecules and oligonucleotides of the disclosed subject matter include segments of DNA, or their complements, which include the c.1082G>A polymorphism in Abraxas. The segments can be between 5 and 250 bases, and, in specific embodiments, are between 5-10, 5-20, 10-20, 10-50, 20-50 or 10-100 bases. The polymorphic site can occur within any position of the segment. The segments can be from any of the allelic forms of DNA.

As used herein, the terms “nucleotide”, “base” and “nucleic acid” are intended to be equivalent. The terms “nucleotide sequence”, “nucleic acid sequence”, “nucleic acid molecule” and “segment” are intended to be equivalent.

“Hybridization probes” are oligonucleotides which bind in a base-specific manner to a complementary strand of nucleic acid. Such probes include peptide nucleic acids, as described in Nielsen et al., Science 254, 1497-1500 (1991). Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range from about 5 nucleotides to about 30 nucleotides in length. For example, probes and primers can be 5, 6, 8, 10, 12, 14, 16, 18, 20, 21, 22, 23, 24, 25, 26, 27, 28 or 30 nucleotides in length. The probe or primer can overlap at least one polymorphic site occupied by any of the possible variant nucleotides. The nucleotide sequence can correspond to the coding sequence of the allele or to the complement of the coding sequence of the allele.

As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

As used herein, “linkage” describes the tendency of genes, alleles, loci or genetic markers to be inherited together as a result of their location on the same chromosome. It can be measured by percent recombination between the two genes, alleles, loci or genetic markers.

As used herein, “polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic fauns are designated as alternative or variant alleles. The allelic form occurring most frequently in a selected population is sometimes referred to as the wild type form. Diploid organisms may be homozygous or heterozygous for allelic forms. A dialleic or biallelic polymorphism has two forms. A triallelic polymorphism has three forms.

A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base.

The disclosed subject matter also relates to nucleic acid molecules which hybridize to the variant alleles identified herein (or their complements) and which also include the variant nucleotide at the SNP site. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

The term “Abraxas” as used herein refers to a polypeptide that is a component of the BRCA1-A complex, and interacts with the BRCA1 BRCT (BRCA1 carboxyl-terminal) repeats and contributes to BRCA1-dependent DNA damage responses. In one non-limiting embodiment, the Abraxas is human Abraxas. In one embodiment, human Abraxas is encoded by the human Abraxas gene (GenBank Accession number EF531340; SEQ ID NO:1; FIG. 8), a nucleic acid which encodes the human Abraxas polypeptide. Alternatively, Abraxas can be encoded by any nucleic acid molecule or fragment having at least about 60%, preferably at least about 70, 80 or 85%, more preferably at least about 90%, even more preferably at least about 95%, and most preferably at least about 98% identity to the human Abraxas gene.

The percent identity of two nucleotide or amino acid sequences can be determined by aligning the sequences for optimal comparison purposes (e.g., can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, preferably at least 40%, more preferably at least 60%, and even more preferably at least 70%, 80% or 90% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:389-3402 (1997). When utilizing BLAST and Gapped BLAST programs, the default parameters of the respective programs (e.g., NBLAST) can be used. See www.ncbi.nlm.nih.gov. In one embodiment, parameters for sequence comparison can be set at score=100, wordlength=12, or can be varied (e.g., W=5 or W=20).

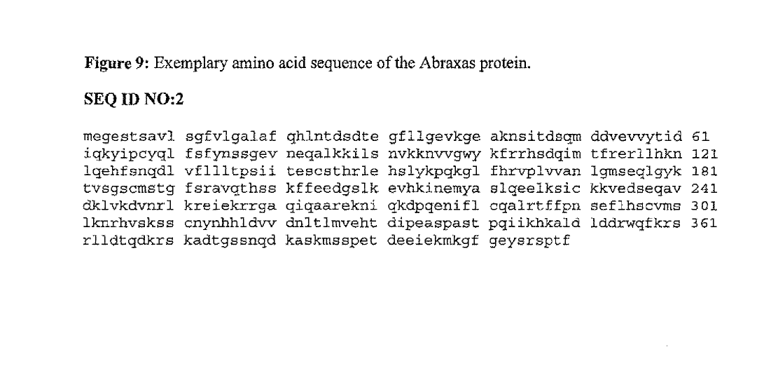

In other non-limiting embodiments, Abraxas may be characterized as having an amino acid sequence depicted in SEQ ID NO:2 (GenBank Accession number: ABP87396.1; FIG. 9) or any other amino acid sequence having at least about 60%, preferably at least about 70, 80 or 85%, more preferably at least about 90%, even more preferably at least about 95%, and most preferably at least about 98% identity to human Abraxas.

Abraxas can be a recombinant Abraxas polypeptide encoded by a recombinant nucleic acid, for example, a non-naturally-ocurring recombinant DNA molecule, or may be of natural origin.

In a non-limiting embodiment, the Abraxas mutation results in the expression of an Abraxas isoform that has an altered function. For example, the Arg361Gln mutation in Abraxas impairs nuclear localization of the protein, and disrupts BRCA1 DNA damage repair function, but does not affect the binding between Abraxas and BRCA.

In preferred non-limiting embodiments, the Abraxas mutation is a G>A point mutation at nucleotide 1082 of the Abraxas gene, resulting in a mutant Abraxas nucleic acid sequence, where nucleotide 1082 is in exon 9 of the Abraxas gene, and the Abraxas gene is numbered according to the Abraxas gene defined by GenBank Accession number EF531340 (SEQ ID NO:1; FIG. 8). The Abraxas polypeptide isofoun produced by the 1082 G>A mutation has an Arg to Gin mutation at amino acid residue 361 of the Abraxas protein defined by GenBank accession number ABP87396.1 (SEQ ID NO:2; FIG. 9), which impairs nuclear localization of the protein, and disrupts BRCA1 DNA damage repair function, but does not affect the binding between Abraxas and BRCA.

The term “cDNA” refers to DNA prepared using messenger RNA (mRNA) as template. The advantage of using a cDNA, as opposed to genomic DNA, is that the cDNA primarily contains coding sequences of the corresponding protein. There may be times when the full or partial genomic sequence is preferred.

A nucleic acid may be contained in a host cell, in some cases, capable of expressing the product of that nucleic acid. In addition to diagnostic and therapeutic considerations, cells expressing nucleic acids of the disclosed subject matter may prove useful in the context of screening for agents that induce, repress, inhibit, augment, interfere with, block, abrogate, stimulate or enhance the expression, distribution, turnover, or detectability of Abraxas transcripts, Abraxas polypeptides, or desired fragments of Abraxas transcripts or Abraxas polypeptides.

As used herein, a “therapeutic agent”, “treatment”, or “drug”, may include any agent used in the treatment (including therapeutic or preventive treatment) of cancer, particularly breast cancer, such as, for example, surgical intervention, chemotherapy with a given drug or drug combination, and/or radiation therapy.

### Isolated Nucleic Acid Molecules

The disclosed subject matter provides isolated nucleic acid molecules that contain the 1082G>A polymorphism in the Abraxas gene. Isolated nucleic acid molecules may optionally encode a full-length variant protein or fragment thereof. The isolated nucleic acid molecules can also include probes and primers (which are described in greater detail below), and isolated full-length genes, transcripts, cDNA molecules, and fragments thereof, which may be used for such purposes as expressing an encoded protein.

As used herein, an “isolated nucleic acid molecule” generally is one that contains a SNP of the disclosed subject matter or one that hybridizes to such molecule such as a nucleic acid with a complementary sequence, and is separated from most other nucleic acids present in the natural source of the nucleic acid molecule. As used herein, “a non-naturally occuring nucleic acid molecule” generally is one that contains a SNP of the disclosed subject matter or one that hybridizes to such a molecule, such as a nucleic acid with a complementary sequence, but which does not correspond to a naturally occuring molecule, e.g., it can be a molecule prepared by recombinant nucleic acid technology, chemical synthesis, or other synthetic means such as polymerase chain reaction (PCR).

Generally, an isolated SNP-containing nucleic acid molecule includes the 1082G>A polymorphism position disclosed herein with flanking nucleotide sequences on either side of the SNP position. A flanking sequence can include nucleotide residues that are naturally associated with the SNP site and/or heterologous nucleotide sequences. Preferably, the flanking sequence is up to about 500, 300, 100, 60, 50, 30, 25, 20, 15, 10, 8, or 4 nucleotides (or any other length in-between) on either side of a SNP position, or as long as the full-length gene or entire protein-coding sequence (or any portion thereof such as an exon), especially if the SNP-containing nucleic acid molecule is to be used to produce a protein or protein fragment.

For full-length genes and entire protein-coding sequences, a SNP flanking sequence can be, for example, up to about 5 KB, 4 KB, 3 KB, 2 KB, 1 KB on either side of the SNP. Furthermore, in such instances the isolated nucleic acid molecule includes exonic sequences (including protein-coding and/or non-coding exonic sequences), but may also include intronic sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can include, for example, a full-length gene or transcript, such as a gene isolated from genomic DNA (e.g., by cloning or PCR amplification), a cDNA molecule, or an mRNA transcript molecule. Furthermore, fragments of such full-length genes and transcripts that contain the SNP disclosed herein are also encompassed by the disclosed subject matter. Such fragments may be used, for example, to express any part of a protein, such as a particular functional domain or an antigenic epitope. A fragment typically includes a contiguous nucleotide sequence at least about 8 or more nucleotides, more preferably at least about 12 or more nucleotides, and even more preferably at least about 16 or more nucleotides. Furthermore, a fragment could include at least about 18, 20, 22, 25, 30, 40, 50, 60, 80, 100, 150, 200, 250 or 500 nucleotides in length (or any other number in between). The length of the fragment will be based on its intended use. For example, the fragment can encode epitope-bearing regions of a variant peptide or regions of a variant peptide that differ from the normal/wild-type protein, or can be useful as a polynucleotide probe or primer.

An isolated nucleic acid molecule of the disclosed subject matter further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195 and 4,683,202; PCR Technology: Principles and Applications for DNA Amplification, ed. H. A. Erlich, Freeman Press, NY, N.Y. (1992)), ligase chain reaction (LCR) (Wu and Wallace, Genomics 4:560 (1989); Landegren et al., Science 241:1077 (1988)), strand displacement amplification (SDA) (U.S. Pat. Nos. 5,270,184 and 5,422,252), transcription-mediated amplification (TMA) (U.S. Pat. No. 5,399,491), linked linear amplification (LLA) (U.S. Pat. No. 6,027,923) and the like, and isothermal amplification methods such as nucleic acid sequence based amplification (NASBA) and self-sustained sequence replication (Guatelli et al., Proc Natl Acad Sci USA 87:1874 (1990)). Based on such methodologies, a person skilled in the art can readily design primers in any suitable regions 5′ and 3′ to a SNP disclosed herein. Such primers may be used to amplify DNA of any length so long that it contains the SNP of interest in its sequence.

As used herein, an “amplified polynucleotide” of the disclosed subject matter is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In an embodiment of the disclosed subject matter, an amplified polynucleotide is at least about 30 nucleotides in length. In another embodiment, an amplified polynucleotide is at least about 32, 40, 45, 50, or 60 nucleotides in length. In yet another preferred embodiment of the disclosed subject matter, an amplified polynucleotide is at least about 100, 200, 300, 400, or 500 nucleotides in length. While the total length of an amplified polynucleotide of the disclosed subject matter can be as long as an exon, an intron or the entire gene where the SNP of interest resides, an amplified product is typically up to about 1,000 nucleotides in length (although certain amplification methods may generate amplified products greater than 1000 nucleotides in length). More preferably, an amplified polynucleotide is not greater than about 600-700 nucleotides in length. It is understood that irrespective of the length of an amplified polynucleotide, a SNP of interest may be located anywhere along its sequence.

The disclosed subject matter provides isolated nucleic acid molecules that include, consist of, or consist essentially of one or more polynucleotide sequences that contain the SNP disclosed herein, complements thereof, and SNP-containing fragments thereof. A nucleic acid molecule consists essentially of a nucleotide sequence when such a nucleotide sequence is present with only a few additional nucleotide residues in the final nucleic acid molecule.

The isolated nucleic acid molecules can encode mature proteins plus additional amino or carboxyl-terminal amino acids or both, or amino acids interior to the mature peptide (when the mature form has more than one peptide chain, for instance). Such sequences may play a role in processing of a protein from precursor to a mature form, facilitate protein trafficking, prolong or shorten protein half-life, or facilitate manipulation of a protein for assay or production. As generally is the case in situ, the additional amino acids may be processed away from the mature protein by cellular enzymes.

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof. Sambrook and Russell, Molecular Cloning: A Laboratory Manual, Cold Spring Harbor Press, N.Y. (2000). Furthermore, isolated nucleic acid molecules, particularly SNP detection reagents such as probes and primers, can also be partially or completely in the form of one or more types of nucleic acid analogs, such as peptide nucleic acid (PNA). U.S. Pat. Nos. 5,539,082; 5,527,675; 5,623,049; and 5,714,331. The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art. See, e.g., Corey, “Peptide nucleic acids: expanding the scope of nucleic acid recognition,” Trends Biotechnol 15 (6):224-9 (June 1997), and Hyrup et al., “Peptide nucleic acids (PNA): synthesis, properties and potential applications,” Bioorg Med Chem 4 (1):5-23 (January 1996). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System, and the sequence information provided herein.

The disclosed subject matter encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting the SNPs identified herein. Furthermore, kits/systems (such as beads, arrays, etc.) that include these analogs are also encompassed herein.

The disclosed subject matter further provides nucleic acid molecules that encode fragments of the variant polypeptides disclosed herein as well as nucleic acid molecules that encode obvious variants of such variant polypeptides. Such nucleic acid molecules have sequences that are naturally occurring, such as paralogs (different locus) and orthologs (different organism), or may be non-naturally occuring, such as those constructed by recombinant DNA methods, by chemical synthesis, or by other synthetic means such as PCR. Non-naturally occurring variants may be made by mutagenesis techniques, including those applied to nucleic acid molecules, cells, or organisms. Accordingly, the variants can contain nucleotide substitutions, deletions, inversions and insertions (in addition to the SNP disclosed herein). Variation can occur in either or both the coding and non-coding regions. The variations can produce conservative and/or non-conservative amino acid substitutions.

Further variants of the nucleic acid molecules disclosed herein, such as naturally occurring allelic variants (as well as orthologs and paralogs) and synthetic variants produced by mutagenesis techniques, can be identified and/or produced using methods well known in the art. Such further variants can include a nucleotide sequence that shares at least 70-80%, 80-85%, 85-90%, 91%, 92%, 93%, 94%, 95%, 96%, 97%, 98%, or 99% sequence identity with a nucleic acid sequence disclosed herein (e.g., SEQ ID NO:1; FIG. 8) (or a fragment thereof) and that includes a novel SNP allele disclosed herein. Further, variants can include a nucleotide sequence that encodes a polypeptide that shares at least 70-80%, 80-85%, 85-90%, 91%, 92%, 93%, 94%, 95%, 96%, 97%, 98%, or 99% sequence identity with a polypeptide sequence disclosed herein (e.g., SEQ ID NO:2; FIG. 9) (or a fragment thereof) and that includes a novel SNP allele disclosed herein.

### Uses of Nucleic Acid Molecules

The nucleic acid molecules of the disclosed subject matter have a variety of uses, especially for the diagnosis, prognosis, and treatment, of cancer, e.g., breast cancer. For example, the nucleic acid molecules of the disclosed subject matter are useful for predicting an individual's risk for developing cancer, e.g., breast cancer, for prognosing the progression of cancer, e.g., breast cancer, in an individual, in evaluating the likelihood of an individual who has cancer, e.g., breast cancer (or who is at increased risk for cancer, e.g., breast cancer) of responding to treatment (or prevention) of cancer, e.g., breast cancer with a particular therapy. For example, the nucleic acid molecules are useful as hybridization probes, such as for genotyping SNPs in messenger RNA, transcript, cDNA, genomic DNA, amplified DNA or other nucleic acid molecules, and for isolating full-length cDNA and genomic clones encoding the mutant polypeptides encoded by the Abraxas gene containing the SNP described herein.

### SNP Detection Reagents

In a specific aspect of the disclosed subject matter, the SNP disclosed herein can be used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP-containing nucleic acid molecule by complementary base-pairing in a sequence specific manner, and discriminates the target variant sequence from other nucleic acid sequences such as an art-known form in a test sample. An example of a detection reagent is a non-naturally ocurring nucleic acid probe that hybridizes to a target nucleic acid containing the SNP disclosed herein. In a preferred embodiment, such a probe can differentiate between nucleic acids having a particular nucleotide (allele) at the target SNP position from other nucleic acids that have a different nucleotide at the same target SNP position. In addition, a detection reagent may hybridize to a specific region 5′ and/or 3′ to the SNP position.

Another example of a detection reagent is a non-naturally ocurring nucleic acid primer that acts as an initiation point of nucleotide extension along a complementary strand of a target polynucleotide. The SNP sequence information provided herein is also useful for designing primers, e.g. allele-specific primers, to amplify (e.g., using PCR) the SNP of the disclosed subject matter. In one non-limiting embodiment, primers useful for amplifying the SNP have the sequences of SEQ ID NOs:21 and 22.

In one preferred embodiment of the disclosed subject matter, a SNP detection reagent is an isolated or synthetic DNA or RNA polynucleotide probe or primer or PNA oligomer, or a combination of DNA, RNA and/or PNA, that hybridizes to a segment of a target nucleic acid molecule containing the SNP disclosed herein. A detection reagent in the form of a non-naturally occuring polynucleotide may optionally contain modified base analogs, intercalators or minor groove binders. Multiple detection reagents such as probes may be, for example, affixed to a solid support (e.g., arrays or beads) or supplied in solution (e.g. probe/primer sets for enzymatic reactions such as PCR, RT-PCR, TaqMan assays, or primer-extension reactions) to form a SNP detection kit.

A probe or primer typically is a substantially purified oligonucleotide or DNA oligomer. Such oligonucleotide typically includes a region of complementary nucleotide sequence that hybridizes under stringent conditions to at least about 8, 10, 12, 16, 18, 20, 22, 25, 30, 40, 50, 55, 60, 65, 70, 80, 90, 100, 120 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

It will be apparent to one of skill in the art that such primers and probes are directly useful as reagents for genotyping the SNP of the disclosed subject matter, and can be incorporated into any kit/system format.

In order to produce a probe or primer specific for a target SNP-containing sequence, the gene/transcript and/or context sequence surrounding the SNP of interest is typically examined using a computer algorithm that starts at the 5′ or at the 3′ end of the nucleotide sequence. Typical algorithms will then identify oligomers of defined length that are unique to the gene/SNP context sequence, have a GC content within a range suitable for hybridization, lack predicted secondary structure that may interfere with hybridization, and/or possess other desired characteristics or that lack other undesired characteristics.

A primer or probe of the disclosed subject matter is typically at least about 8 nucleotides in length. In one embodiment, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length.

For analyzing SNPs, it can be appropriate to use oligonucleotides specific for alternative SNP alleles. Such oligonucleotides that detect single nucleotide variations in target sequences may be referred to by such terms as “allele-specific oligonucleotides,” “allele-specific probes,” or “allele-specific primers.” The design and use of allele-specific probes for analyzing polymorphisms is described in, e.g., Mutation Detection: A Practical Approach, Cotton et al., eds., Oxford University Press (1998); Saiki et al., Nature 324:163-166 (1986); Dattagupta, EP235,726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the condition under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: prehybridization with a solution containing 5× standard saline phosphate EDTA (SSPE), 0.5% NaDodSO₄ (SDS) at 55° C., and incubating probe with target nucleic acid molecules in the same solution at the same temperature, followed by washing with a solution containing 2×SSPE, and 0.1% SDS at 55° C. or room temperature.

Moderate stringency hybridization conditions may be used for allele-specific primer extension reactions with a solution containing, e.g., about 50 mM KCI at about 46° C. Alternatively, the reaction may be carried out at an elevated temperature such as 60° C. In another embodiment, a moderately stringent hybridization condition suitable for oligonucleotide ligation assay (OLA) reactions where two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer. In a specific preferred embodiment that is particularly suitable for use in a oligonucleotide ligation assay (U.S. Pat. No. 4,988,617), the 3′ most nucleotide of the probe aligns with the SNP position in the target sequence.

Oligonucleotide probes and primers may be prepared by methods well known in the art. Chemical synthetic methods include, but are not limited to, the phosphotriester method described by Narang et al., Methods in Enzymology 68:90 (1979); the phosphodiester method described by Brown et al., Methods in Enzymology 68:109 (1979); the diethylphosphoamidate method described by Beaucage et al., Tetrahedron Letters 22:1859 (1981); and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position. Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity. Gibbs, Nucleic Acid Res 17:2427-2448 (1989). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). This PCR-based assay can be utilized as part of the TaqMan assay, described below.

In another embodiment, a primer contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

In another embodiment, a SNP detection reagent of the disclosed subject matter is labeled with a fluorogenic reporter dye that emits a detectable signal. While the preferred reporter dye is a fluorescent dye, any reporter dye that can be attached to a detection reagent such as an oligonucleotide probe or primer is suitable for use in the disclosed subject matter. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment, the detection reagent may be further labeled with a quencher dye such as Tamra, especially when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., PCR Method Appl 4:357-362 (1995); Tyagi et al., Nature Biotechnology 14:303-308 (1996); Nazarenko et al., Nuc′ Acids Res 25:2516-2521 (1997); U.S. Pat. Nos. 5,866,336 and 6,117,635.

The detection reagents of the disclosed subject matter may also contain other labels, including but not limited to, biotin for streptavidin binding, hapten for antibody binding, and oligonucleotide for binding to another complementary oligonucleotide.

The disclosed subject matter also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the Y-most end of the primer extension product, and in which the ddNTP is a nucleotide of a SNP disclosed herein, is a composition that is specifically herein). Thus, reagents that bind to a nucleic acid molecule in a region adjacent to a SNP site and that are used for assaying the SNP site, even though the bound sequences do not necessarily include the SNP site itself, are also contemplated by the disclosed subject matter.

In certain embodiments the reagents and techniques described herein will be directed to performance of “Next Generation Sequencing.” (See, e.g., Srivatsan et al., PLoS Genet 4: e100139 (2008); Rasmussen et al., Nature 463:757-762 (2010); Li et al., Nature 463: 311-317 (2010); Pelak et al., PLoS Genet 6: e1001111 (2010); Ram et al., Syst Biol Reprod Med (57(3):117-118 (2011); McEllistrem, Future Microbiol 4: 857-865 (2009); Lo et al., Clin Chem 55: 607-608 (2009); Robinson, Genome Biol 11:144 (2010); and Araya et al., Trends Biotechnology doi10.1016.j.tibtech.2011.04.003 (2011)). For example, but not by way of limitation, such techniques can involve the fragmentation of a genomic nucleic acid sample followed by parallel sequencing of those fragments and the alignment of the sequenced fragments to reconstruct the original sequence. In certain embodiments, the genomic nucleic acid of interest is sheared into fragments and “adapters” (short nucleic acids of known sequence) are ligated to the fragments. In certain embodiments, these adaptor-modified fragments can be enriched via PCR. In certain embodiments the adaptor-modified fragments (and amplified copies thereof, if present) are then flowed across a flow cell where the fragments are allowed to hybridize to primers immobilized on the surface of the cell. The fragments are then amplified by isothermal bridge amplification into a cluster consisting of thousands of molecules identical to the original. Sequencing primers can then be hybridized to the ends of one strand of the clusters, and reversibly blocked & labeled nucleotides can be added. The addition of each particular nucleotide can be identified by the label, then the label can be removed and the nucleotide un-blocked so that another blocked & labeled nucleotide can be added to identify the next position in the nucleic acid sequence. Once the desired number of rounds of addition, detection, and unblocking occur, the resulting sequences can be aligned.

### SNP Detection Kits and Systems

A person skilled in the art will recognize that, based on the SNP and associated sequence information disclosed herein, detection reagents can be developed and used to assay the SNP of the disclosed subject matter individually or in combination with other SNPs, and such detection reagents can be readily incorporated into one of the established kit or system formats which are well known in the art.

The terms “kits” and “systems,” as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the disclosed subject matter further provides SNP detection kits and systems, including but not limited to, packaged probe and primer sets (e.g. TaqMan probe/primer sets), arrays/microarrays of nucleic acid molecules, and beads that contain one or more probes, primers, or other detection reagents for detecting one or more SNPs of the disclosed subject matter. In one non-limiting embodiment, the kit includes a pair of oligonucleotide primers having the sequences of SEQ ID NOs: 21 and 22.

The kits/systems can optionally include various electronic hardware components; for example, arrays (“DNA chips”) and microfluidic systems (“lab-on-a-chip” systems) provided by various manufacturers typically include hardware components. Other kits/systems (e.g., probe/primer sets) may not include electronic hardware components, but may include of, for example, one or more SNP detection reagents (along with, optionally, other biochemical reagents) packaged in one or more containers.

In some embodiments, a SNP detection kit typically contains one or more detection reagents and other components (e.g. a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can include instructions for using the kit to detect the SNP-containing nucleic acid molecule of interest.

In one embodiment, kits are provided which contain the necessary reagents to carry out one or more assays to detect one or more SNPs disclosed herein. In another embodiment, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is the SNP of the disclosed subject matter. In some kits/systems, the allele-specific probes are immobilized to a substrate such as an array or bead.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in Chee et al., U.S. Pat. No. 5,837,832 and PCT application WO95/11995; D. J. Lockhart et al., Nat Biotech 14:1675-1680 (1996); and M. Schena et al., Proc Natl Acad Sci 93:10614-10619 (1996), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics,” Biotechnol Annu Rev 8:85-101 (2002); Sosnowski et al., “Active microelectronic array system for DNA hybridization, genotyping and pharmacogenomic applications,” Psychiatr Genet 12 (4):181-92 (December 2002); Heller, “DNA microarray technology: devices, systems, and applications,” Annu Rev Biomed Eng 4:129-53 (2002); Epub Mar. 22, 2002; Kolchinsky et al., “Analysis of SNPs and other genomic variations using gel-based chips,” Hum Mutat 19 (4):343-60 (April 2002); and McGall et al., “High-density genechip oligonucleotide probe arrays,” Adv Biochem Eng Biotechnol 77:21-42 (2002).

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology 6.3.1-6.3.6, John Wiley & Sons, N.Y. (1989).

In other embodiments, the arrays are used in conjunction with chemiluminescent detection technology. The following patents and patent applications, which are all hereby incorporated by reference, provide additional information pertaining to chemiluminescent detection. U.S. patent applications that describe chemiluminescent approaches for microarray detection: Ser. Nos. 10/620,332 and 10/620,333. U.S. patents that describe methods and compositions of dioxetane for performing chemiluminescent detection: U.S. Pat. Nos. 6,124,478; 6,107,024; 5,994,073; 5,981,768; 5,871,938; 5,843,681; 5,800,999 and 5,773,628. And the U.S. published application that discloses methods and compositions for microarray controls: US2002/0110828.

In one embodiment, a nucleic acid array can include an array of probes of about 15-25 nucleotides in length. In further embodiments, a nucleic acid array can include any number of probes, in which at least one probe is capable of detecting the SNP disclosed herein, and/or at least one probe includes a fragment of one of the sequences encompassing the SNP disclosed herein, and sequences complementary thereto, the fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) the SNP disclosed herein. In some embodiments, the nucleotide complementary to the SNP site is within 5, 4, 3, 2, or 1 nucleotide from the center of the probe, more preferably at the center of the probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the disclosed subject matter provides methods of identifying the SNPs disclosed herein in a test sample. Such methods typically involve incubating a test sample of nucleic acids with an array comprising one or more probes corresponding the SNP of the disclosed subject matter and other (known) SNPs, and assaying for binding of a nucleic acid from the test sample with one or more of the probes.

A SNP detection kit/system of the disclosed subject matter can include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue or tumor specimens. Methods of preparing nucleic acids, proteins, and cell extracts are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM 6700 sample preparation system, and Roche Molecular Systems' COBAS AmpliPrep System.

Another form of kit contemplated by the disclosed subject matter is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting the SNP of the disclosed subject matter, one or more containers which contain wash reagents (such as phosphate buffered saline, Tris-buffers, etc.), and one or more containers which contain the reagents used to reveal the presence of the bound probe or other SNP detection reagents. The kit can optionally further include compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art. See, e.g., Weigl et al, “Lab-on-a-chip for drug development,” Adv Drug Deliv Rev 55 (3):349-77 (February 2003). In such microfluidic devices, the containers may be referred to as, for example, microfluidic “compartments,” “chambers,” or “channels.”

Microfluidic devices, which may also be referred to as “lab-on-a-chip” systems, biomedical micro-electro-mechanical systems (bioMEMs), or multicomponent integrated systems, are exemplary kits/systems of the disclosed subject matter for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect the SNP of the disclosed subject matter. One example of a microfluidic system is disclosed in U.S. Pat. No. 5,589,136, which describes the integration of PCR amplification and capillary electrophoresis in chips. Exemplary microfluidic systems include a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073, Dubrow et al., and U.S. Pat. No. 6,156,181, Parce et al.

For genotyping SNPs, an exemplary microfluidic system may integrate, for example, nucleic acid amplification, primer extension, capillary electrophoresis, and a detection method such as laser induced fluorescence detection. In an exemplary process for using such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted SNP. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. Such an exemplary microchip can be used to process, for example, at least 96 to 384 samples, or more, in parallel.

### Abraxas SNP Genotyping Methods

The process of determining which nucleotide is present at the SNP position described herein, for either or both alleles, can be referred to by such phrases as SNP genotyping, determining the “identity” of a SNP, determining the “content” of a SNP, or determining which nucleotide(s)/allele(s) is/are present at the SNP position. Thus, these terms can refer to detecting a single allele (nucleotide) at a SNP position or can encompass detecting both alleles (nucleotides) at a SNP position (such as to determine the homozygous or heterozygous state of a SNP position). Furthermore, these terms may also refer to detecting an amino acid residue encoded by a SNP (such as alternative amino acid residues that are encoded by different codons created by alternative nucleotides at a SNP position).

The disclosed subject matter provides methods of SNP genotyping, such as for use in evaluating an individual's risk for developing cancer, e.g., breast cancer, for evaluating an individual's prognosis for disease severity and recovery, for implementing a preventive or treatment regimen for an individual based on that individual having an increased susceptibility for developing cancer, in evaluating an individual's likelihood of responding to a therapeutic treatment for cancer, e.g., chemotherapy, in selecting a treatment or preventive regimen (e.g., in deciding whether or not to administer a particular therapeutic agent to an individual having cancer, e.g., breast cancer, or who is at increased risk for developing cancer, e.g., breast cancer in the future), or in formulating or selecting a particular treatment or preventive regimen such as dosage and/or frequency of administration of a therapeutic agent or choosing which form/type of a therapeutic agent to be administered, such as a particular pharmaceutical composition or compound, etc.), or selecting individuals for a clinical trial of a therapeutic or preventative agent (e.g., selecting individuals to participate in the trial who are most likely to respond positively to a therapeutic agent and/or excluding individuals from the trial who are unlikely to respond positively to a therapeutic agent based on their SNP genotype(s), or selecting individuals who are unlikely to respond positively to a particular therapeutic agent based on their SNP genotype(s) to participate in a clinical trial of another therapeutic agent that may benefit them), etc.

In certain embodiments, the samples described herein can be derived from any tissues, cells and/or cells in biological fluids from, for example, a mammal or human to be tested. Sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue or tumor specimens.

Nucleic acid samples can be genotyped to determine which allele is present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput,” Pharmacogenomics J 3 (2):77-96 (2003); Kwok et al., “Detection of single nucleotide polymorphisms,” Curr Issues Mol Biol 5 (2):43-60 (April 2003); Shi, “Technologies for individual genotyping: detection of genetic polymorphisms in drug targets and disease genes,” Am J Pharmacogenomics 2 (3):197-205 (2002); and Kwok, “Methods for genotyping single nucleotide polymorphisms,” Annu Rev Genomics Hum Genet 2:235-58 (2001). Exemplary techniques for high-throughput SNP genotyping are described in Mamellos, “High-throughput SNP analysis for genetic association studies,” Curr Opin Drug Discov Devel 6 (3):317-21 (May 2003).

Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, denaturing gradient gel electrophoresis, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol 217:286-295 (1992)), comparison of the electrophoretic mobility of variant and wild type nucleic acid molecules (Orita et al, PNAS 86:2766 (1989); Cotton et al, Mutat Res 285:125-144 (1993); and Hayashi et al., Genet Anal Tech Appl 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In one embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the disclosed subject matter are useful in, for example, screening for individuals who are susceptible to developing cancer (particularly breast cancer) and related pathologies, or in screening individuals who have cancer (particularly breast cancer) (or who are susceptible to cancer (particularly breast cancer) for their likelihood of responding to a particular treatment (e.g., chemotherapy)). These probes and primers can be readily incorporated into a kit format. The disclosed subject matter also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another method for genotyping the SNPs can be the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA. The following U.S. patents describe OLA strategies for performing SNP detection: U.S. Pat. Nos. 6,027,889; 6,268,148; 5,494,810; 5,830,711 and 6,054,564. WO 97/31256 and WO 00/56927 describe OLA strategies for performing SNP detection using universal arrays, where a zipcode sequence can be introduced into one of the hybridization probes, and the resulting product, or amplified product, hybridized to a universal zip code array. U.S. application Ser. No. 01/17,329 (and Ser. No. 09/584,905) describes OLA (or LDR) followed by PCR, where zipcodes are incorporated into OLA probes, and amplified PCR products are determined by electrophoretic or universal zipcode array readout. U.S. applications 60/427,818, 60/445,636, and 60/445,494 describe SNPIex methods and software for multiplexed SNP detection using OLA followed by PCR, where zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry,” Rapid Commun Mass Spectrom 17 (11):1195-202 (2003).

The following references provide further information describing mass spectrometry-based methods for SNP genotyping: Bocker, “SNP and mutation discovery using base-specific cleavage and MALDI-TOF mass spectrometry,” Bioinformatics 19 Suppl 1:144-153 (July 2003); Storm et al., “MALDI-TOF mass spectrometry-based SNP genotyping,” Methods Mol Biol 212:241-62 (2003); Jurinke et al., “The use of Mass ARRAY technology for high throughput genotyping,” Adv Biochem Eng Biotechnol 77:57-74 (2002); and Jurinke et al., “Automated genotyping using the DNA MassArray technology,” Methods Mol Biol 187:179-92 (2002).

SNPs can also be scored by direct DNA sequencing. A variety of automated sequencing procedures can be utilized (e.g. Biotechniques 19:448 (1995)), including sequencing by mass spectrometry. See, e.g., PCT International Publication No. WO 94/16101; Cohen et al., Adv Chromatogr 36:127-162 (1996); and Griffin et al, Appl Biochem Biotechnol 38:147-159 (1993). The nucleic acid sequences of the disclosed subject matter enable one of ordinary skill in the art to readily design sequencing primers for such automated sequencing procedures. Commercial instrumentation, such as the Applied Biosystems 377, 3100, 3700, 3730, and 3730x1 DNA Analyzers (Foster City, Calif.), is commonly used in the art for automated sequencing.

Other methods that can be used to genotype the SNPs of the disclosed subject matter include single-strand conformational polymorphism (SSCP), and denaturing gradient gel electrophoresis (DGGE). Myers et al., Nature 313:495 (1985). SSCP identifies base differences by alteration in electrophoretic migration of single stranded PCR products, as described in Orita et al., Proc. Nat. Acad. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel. PCR Technology: Principles and Applications for DNA Amplification Chapter 7, Erlich, ed., W.H. Freeman and Co, N.Y. (1992).

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

SNP genotyping can include, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described herein. Examples of such applications include, but are not limited to, SNP-disease association analysis, disease predisposition screening, disease diagnosis, disease prognosis, disease progression monitoring, determining therapeutic strategies based on an individual's genotype (“pharmacogenomics”), developing therapeutic agents based on SNP genotypes associated with a disease or likelihood of responding to a drug, stratifying patient populations for clinical trials of a therapeutic, preventive, or diagnostic agent, and human identification applications such as forensics.

### Detection of Polymorphisms in Abraxas Polypeptides

The disclosed subject matter also relates to a method for predicting the likelihood that an individual will develop cancer, e.g., breast cancer, or for aiding in the diagnosis of cancer, e.g., breast cancer, including obtaining a biological sample comprising Abraxas protein or relevant portion thereof from an individual to be assessed and determining the amino acid present at one or more of amino acid positions 361 of the Abraxas gene product (e.g., as exemplified by SEQ ID NO: 2). As used herein, the term “relevant portion” of the Abraxas protein is intended to encompass any portion of the protein which includes the polymorphic amino acid position. The presence of one or more of a Gin (the variant amino acid) at position 361 of SEQ ID NO: 2 indicates that the individual has a greater likelihood of developing cancer, e.g., breast cancer, than if that individual had the reference amino acid at this position. Conversely, the presence of one or more of an Arg (the reference amino acid) at position 361 of SEQ ID NO: 2 indicates that the individual has a reduced likelihood of developing cancer, e.g., breast cancer, than if that individual had the variant amino acid at this positions.

In this embodiment of the disclosed subject matter, the biological sample contains protein molecules from the test subject. In vitro techniques for detection of protein include enzyme linked immunosorbent assays (ELISAs), Western blots, immunoprecipitations and immunofluorescence. Furthermore, in vivo techniques for detection of protein include introducing into a subject a labeled anti-protein antibody. For example, the antibody can be labeled with a radioactive marker whose presence and location in a subject can be detected by standard imaging techniques. Polyclonal and/or monoclonal antibodies that specifically bind to variant gene products but not to corresponding reference gene products, and vice versa, are also provided. Antibodies can be made by injecting mice or other animals with the variant gene product or synthetic peptide fragments thereof comprising the variant portion. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

### Vectors, Host Cells, and Transgenic Animals

The disclosed subject matter further pertains to compositions, e.g., vectors, comprising a nucleotide sequence encoding reference or variant Abraxas gene products. For example, reference genes can be expressed in an expression vector in which a reference gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like.

It is also contemplated that cells can be engineered to express the reference allele of the disclosed subject matter by gene therapy methods. For example, DNA encoding the reference Abraxas gene product, or an active fragment or derivative thereof, can be introduced into an expression vector, such as a viral vector, and the vector can be introduced into appropriate cells in an animal. In such a method, the cell population can be engineered to inducibly or constitutively express active reference Abraxas gene product.

The disclosed subject matter also relates to constructs which include a vector into which a sequence of the disclosed subject matter has been inserted in a sense or antisense orientation. For example, a vector comprising a nucleotide sequence which is antisense to the variant Abraxas allele may be used as an antagonist of the activity of the Abraxas variant allele. Alternatively, a vector comprising a nucleotide sequence of the Abraxas reference allele may be used therapeutically to treat cancer. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the disclosed subject matter is intended to include such other forms of expression vectors, such as viral vectors (e.g., replication defective retroviruses, adenoviruses and adeno-associated viruses) that serve equivalent functions.

Preferred recombinant expression vectors of the disclosed subject matter include a nucleic acid of the disclosed subject matter in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc.

The expression vectors of the disclosed subject matter can be introduced into host cells to thereby produce proteins or peptides, including fusion proteins or peptides, encoded by nucleic acids as described herein. The recombinant expression vectors of the disclosed subject matter can be designed for expression of a polypeptide of the disclosed subject matter in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the disclosed subject matter pertains to host cells into which a recombinant expression vector of the disclosed subject matter has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a nucleic acid of the disclosed subject matter can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

A host cell of the disclosed subject matter, such as a prokaryotic or eukaryotic host cell in culture, can be used to produce (i.e., express) a polypeptide of the disclosed subject matter. Accordingly, the disclosed subject matter further provides methods for producing a polypeptide using the host cells of the disclosed subject matter. In one embodiment, the method includes culturing the host cell of the disclosed subject matter (into which a recombinant expression vector encoding a polypeptide of the disclosed subject matter has been introduced) in a suitable medium such that the polypeptide is produced. In another embodiment, the method further includes isolating the polypeptide from the medium or the host cell.

The host cells of the disclosed subject matter can also be used to produce nonhuman transgenic animals. For example, in one embodiment, a host cell of the disclosed subject matter is a fertilized oocyte or an embryonic stem cell into which a nucleic acid of the disclosed subject matter has been introduced. Such host cells can then be used to create non-human transgenic animals in which exogenous nucleotide sequences have been introduced into their genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the disclosed subject matter can be created by introducing a nucleic acid of the disclosed subject matter into the male pronuclei of a fertilized oocyte, e.g., by microinjection, retroviral infection, and allowing the oocyte to develop in a pseudopregnant female foster animal. The sequence can be introduced as a transgene into the genome of a non-human animal. Intronic sequences and polyadenylation signals can also be included in the transgene to increase the efficiency of expression of the transgene. A tissue-specific regulatory sequence(s) can be operably linked to the transgene to direct expression of a polypeptide in particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA in tissues or cells of the animals. A transgenic founder animal can then be used to breed additional animals carrying the transgene. Moreover, transgenic animals carrying a transgene encoding the transgene can further be bred to other transgenic animals carrying other transgenes.

### Genetic Mapping of Phenotypic Traits

The present disclosed subject matter also includes the identification of a physical linkage between a genetic locus associated with a trait of interest and polymorphic markers that are not associated with the trait, but are in physical proximity with the genetic locus responsible for the trait (the Abraxas SNP described herein) and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992).

The invention includes methods and compositions for diagnosing or predicting increased risk of cancer by identifying SNPs that are linked with the Abraxas SNP described herein.

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27(1991).

Linkage is analyzed by calculation of LOD (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at .theta. to probability of data if loci unlinked. The computed likelihoods are usually expressed as the log 10 of this ratio (i.e., a led score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

### Reports, Programmed Computers, and Systems

The results of a test (e.g., an individual's risk for cancer, such as breast cancer), or an individual's predicted drug responsiveness (e.g., response to chemotherapy), based on assaying the SNP disclosed herein, alone or in combination with other SNPs, and/or an individual's allele/genotype at the SNP disclosed herein, alone or in combination with other SNPs, etc.), and/or any other information pertaining to a test, may be referred to herein as a “report”. A tangible report can optionally be generated as part of a testing process (which may be interchangeably referred to herein as “reporting”, or as “providing” a report, “producing” a report, or “generating” a report).

Examples of tangible reports may include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which may optionally be accessible via the internet (such as a database of patient records or genetic information stored on a computer network server, which may be a “secure database” that has security features that limit access to the report, such as to allow only the patient and the patient's medical practitioners to view the report while preventing other unauthorized individuals from viewing the report, for example). In addition to, or as an alternative to, generating a tangible report, reports can also be displayed on a computer screen (or the display of another electronic device or instrument).

A report can include, for example, an individual's risk for cancer, such as breast cancer, or may just include the allele/genotype that an individual carries at the SNP location disclosed herein, alone or in combination with other SNPs, which may optionally be linked to information regarding the significance of having the allele/genotype at the SNP location(s) (for example, a report on computer readable medium such as a network server may include hyperlink(s) to one or more journal publications or websites that describe the medical/biological implications, such as increased or decreased disease risk, for individuals having a certain allele/genotype at the SNP(s)). Thus, for example, the report can include disease risk or other medical/biological significance (e.g., drug responsiveness, suggested prophylactic treatment, etc.) as well as optionally also including the allele/genotype information, or the report may just include allele/genotype information without including disease risk or other medical/biological significance (such that an individual viewing the report can use the allele/genotype information to determine the associated disease risk or other medical/biological significance from a source outside of the report itself, such as from a medical practitioner, publication, website, etc., which may optionally be linked to the report such as by a hyperlink).

A report can further be “transmitted” or “communicated” (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the interne, by facsimile, and/or by any wired or wireless communication methods known in the art) such as by being retrieved from a database stored on a computer network server, etc.

In certain exemplary embodiments, the disclosed subject matter provides computers (or other apparatus/devices such as biomedical devices or laboratory instrumentation) programmed to carry out the methods described herein. For example, in certain embodiments, the disclosed subject matter provides a computer programmed to receive (i.e., as input) the identity (e.g., the allele or genotype at a SNP) of the SNP disclosed herein, alone or in combination with other SNPs, and provide (i.e., as output) the disease risk (e.g., risk for breast cancer) or other result (e.g., disease diagnosis or prognosis, drug responsiveness, etc.) based on the identity of the SNP(s). Such output (e.g., communication of disease risk, disease diagnosis or prognosis, drug responsiveness, ete.) may be, for example, in the form of a report on computer readable medium, printed in paper form, and/or displayed on a computer screen or other display.

Certain further embodiments of the disclosed subject matter provide a system for determining an individual's cancer risk, or whether an individual will benefit from chemotherapy treatment (or other therapy), or prophylactic treatment. Certain exemplary systems include an integrated “loop” in which an individual (or their medical practitioner) requests a determination of such individual's cancer risk (or drug response), this determination is carried out by testing a sample from the individual, and then the results of this determination are provided back to the requester. For example, in certain systems, a sample (e.g., buccal cells, saliva, blood, etc.) is obtained from an individual for testing (the sample may be obtained by the individual or, for example, by a medical practitioner), the sample is submitted to a laboratory (or other facility) for testing (e.g., determining the genotype of the SNP disclosed herein, alone or in combination with one or more other SNP), and then the results of the testing are sent to the patient (which optionally can be done by first sending the results to an intermediary, such as a medical practitioner, who then provides or otherwise conveys the results to the individual and/or acts on the results), thereby forming an integrated loop system for determining an individual's cancer risk (or drug response, etc.). The portions of the system in which the results are transmitted (e.g., between any of a testing facility, a medical practitioner, and/or the individual) can be carried out by way of electronic or signal transmission (e.g., by computer such as via e-mail or the internet, by providing the results on a website or computer network server which may optionally be a secure database, by phone or fax, or by any other wired or wireless transmission methods known in the art).

In exemplary embodiments, the system is controlled by the individual and/or their medical practitioner in that the individual and/or their medical practitioner requests the test, receives the test results back, and (optionally) acts on the test results to reduce the individual's disease risk, such as by implementing a disease management system.

The various methods described herein, such as correlating the presence or absence of a polymorphism with an altered (e.g., increased or decreased) risk (or no altered risk) for cancer, e.g., breast cancer, can be carried out by automated methods such as by using a computer (or other apparatus/devices such as biomedical devices, laboratory instrumentation, or other apparatus/devices having a computer processor) programmed to carry out any of the methods described herein. For example, computer software (which may be interchangeably referred to herein as a computer program) can perform correlating the presence or absence of a polymorphism in an individual with an altered (e.g., increased or decreased) risk (or no altered risk) for cancer, e.g., breast cancer for the individual. Accordingly, certain embodiments of the disclosed subject matter provide a computer (or other apparatus/device) programmed to carry out any of the methods described herein.

The following Example is offered for the purpose of illustrating the disclosed subject matter and are not to be construed as limitations.

### Example

### Identification of Breast-Cancer Associated Abraxas Mutation

### Materials and Methods

### Familial and Unselected Breast Cancer Cases

Mutation screening of Abraxas was performed on blood DNA samples obtained from 125 breast and breast-ovarian cancer families originating from Northern Finland (23). One index patient from each family was chosen according to the youngest age of breast cancer onset. Inclusion criteria for the 73 high-risk families were as follows: (i) three or more cases of breast cancer, potentially in combination with single ovarian cancer in first- or second-degree relatives, or (ii) two cases of breast or breast and ovarian cancer in first- or second-degree relatives, of which at least one with early disease onset (<35 years), bilateral breast cancer, or multiple primary tumors including breast or ovarian cancer in the same individual. The remaining 52 families were indicative of moderate disease susceptibility, presenting either two cases of breast cancer in first- or second-degree relatives, or breast cancer under the age of 35 (2 cases). Together 15 of the studied index cases had previously been tested positive for known breast cancer associated germline mutations in BRCA1 or BRCA2 (11 cases), TP53 (1 case), and PALB2 (3 cases). All of the biological specimens and clinical information of the familial breast cancer cases investigated were collected at the Oulu University Hospital, with the informed consent of the patients.

For the Abraxas c.1082G>A genotyping and tagging SNP (tagSNP) analysis, DNAs from an unselected cohort of breast cancer patients (N=991) were collected without selection for a family history of breast cancer. This sample set consisted of 544 Northern Finnish cases operated at the Oulu University Hospital during the years 2000-2007 and 447 patients with invasive breast cancer from the Kuopio Breast Cancer Project (KBCP), originating from the province of Northern Savo in Eastern Finland and diagnosed at the Kuopio University Hospital between 1990 and 1995 (37).

Informed consent to participate in the study has been obtained from each patient, and the studies have been approved by the Finnish Ministry of Social Affairs and Health, and appropriate ethical committees of each of the participating University Hospitals.

### Control Cases

Altogether 868 Finnish female control cases were used for genotyping and tagSNP analysis. The Northern Finnish control samples (where the number of studied individuals varied between 88 and 506, depending on the specific analysis) derived from anonymous cancer-free Finnish Red Cross blood donors (age≧45 years) originating from the same geographical region as the studied cancer patient cohort. The age- and area of residence-matched KBCP cohort consisted of DNA from 362 control subjects selected from the National Population Register during the same time period as the unselected breast cancer patients (37). All control individuals were cancer-free at the time of donation of the blood sample. There was no follow-up on donor health status.

### Mutation and TagSNP Analysis

The entire coding region and exon-intron boundaries of the Abraxas gene were screened for germline mutations either by conformation sensitive gel electrophoresis (CSGE, exons 2 to 8) or by direct sequencing (exons 1 and 9). Samples with deviating CSGE patterns or those directly sequenced were analyzed with the Li-Cor IR² 4200-S DNA Analysis system (Li-Cor Inc.) using the SequiTherm EXEL II DNA Sequencing Kit-LC (Epicentre Technologies) or with ABI3730 (ART Perkin Elmer) using the BigDye Terminator v3.1 Cycle Sequencing Kit (Applied Biosystems). Oligonucleotides were designed by using Primer3 software (http://frodo.wi.mit.edu/cgi-bin/primer3/primer3_www.cgi; Table 1).

TABLE 1
Abraxas primers and PCR amplicon details

| | | | Amplicon |
|---|---|---|---|
| | Forward primer | Reverse primer | size |
| Exon | (5′-3′) | (5′-3′) | (bp) |
| 1 | GCCCTCGTCCTCTTGT | AGGGGGAGAGAAGGC | 272 |
| | GTAG | AGAG | |
| | (SEQ ID NO: 3) | (SEQ ID NO: 4) | |
| 2 | CCCCGACTGGTAGCAC | TTGCCCAGCCTCTGT | 323 |
| | ATA | ATTTC | |
| | (SEQ ID NO: 5) | (SEQ ID NO: 6) | |
| 3 | CTTCCTGGCGTGAGGT | CAGAGAAAGAACTGC | 318 |
| | AAAG | TAGGGTTG | |
| | (SEQ ID NO: 7) | (SEQ ID NO: 8) | |
| 4 | GGGCTTTGGTAGTTGG | GCAACCACTTTCAAT | 334 |
| | GTTA | TTCTGG | |
| | (SEQ ID NO: 9) | (SEQ ID NO: 10) | |
| 5 | AAGAAAGCCATTTTAA | TGATGCGACAATATA | 397 |
| | GGTTGTT | TGAGAACAC | |
| | (SEQ ID NO: 11) | (SEQ ID NO: 12) | |
| 6 | TTTTTAAATCTTGTAG | TCCCTTGAATTTTTA | 311 |
| | GGGACAA | TTCTGCTG | |
| | (SEQ ID NO: 13) | (SEQ ID NO: 14) | |
| 7 | GGTTTTGTGGTTGGTT | CATAGCCTTCATTAA | 382 |
| | TTTCT | GCAACTCA | |
| | (SEQ ID NO: 15) | (SEQ ID NO: 16) | |
| 8 | TGCTTGTTACTGACAT | CACCTTTGCACTCCA | 384 |
| | CCTCCA | ACCTA | |
| | (SEQ ID NO: 17) | (SEQ ID NO: 18) | |
| 9^{a} | TTGTCTTAGAATACTG | TGAGTTCCACTGGCC | 1374 |
| | TGGCATATAAA | TATCC | |
| | (SEQ ID NO: 19) | (SEQ ID NO: 20) | |

^{a}Exon 9 has extensive similarity with other genomic contigs, predominantly with chr. 3 and 8. To avoid non-specific priming, a part of intron 8, the coding part of exon 9 and a portion of the 3′ UTR have been amplified in a single fragment, and was directly sequenced. Exon 1 was also analyzed by direct sequencing.

Genotyping of Abraxas c.1082G>A and TagSNPs rs12499395, rs12649417 and rs13125836 was done using MassARRAY mass spectrometer (Sequenom Inc., San Diego, Calif., USA) and iPLEX Gold (Sequenom Inc.) on 384-well plate format (using the primers in Table 2).

TABLE 2
iPlex primers used for Abraxas
c.1082G>A and tagSNP genotyping

| | | Extension |
|---|---|---|
| | | primer |
| Variation | PCR primer sequence* | sequence* |
| c.1082G>A | F: | tgTGTCTTGTGTATCT |
| | acgttggatgCAGCTAGTACAC | AACA |
| | CACAAATC | (SEQ ID NO: 23) |
| | (SEQ ID NO: 21) | |
| | R: | |
| | acgttggatgGATCGTTTGTCT | |
| | TGTGTATC | |
| | (SEQ ID NO: 22) | |
| rs12499395 | F: | CCCAGCTTTGGAC |
| | acgttggatgCATAGTTGACTT | CTAC |
| | AACAGCCC | (SEQ ID NO: 26) |
| | (SEQ ID NO: 24) | |
| | R: | |
| | acgttggatgTCATTGGCTAGG | |
| | AACAGCCC | |
| | (SEQ ID NO: 25) | |
| rs12649417 | F: | cccTCTTCTAAACG |
| | acgttggatgGACAAGCATCAT | TTCTACAGATAAT |
| | ATGGCACC | |
| | (SEQ ID NO: 27) | (SEQ ID NO: 29) |
| | R: | |
| | acgttggatgCATCTTCTAAAC | |
| | GTTCTAC | |
| | (SEQ ID NO: 28) | |
| rs13125836 | F: | TTGGTTACTACTAC |
| | acgttggatgGGATGCTTTATC | CAGTAT |
| | TTGGTTAC | (SEQ ID NO: 32) |
| | (SEQ ID NO: 30) | |
| | R: | |
| | acgttggatgCAAGAGATCTCG | |
| | GTTGTTAG | |
| | (SEQ ID NO: 31) | |

F = Forward primer
R = Reverse primer
*bases in lower case letters are non-templated bases

TagSNPs were selected using the HapMap Genome Browser release 2 (Phase 3, NCBI build 36, bdSNP b126) as of Feb. 23, 2010 (http://hapmap.ncbi.nlm.nih.gov/cgi-perl/gbrowse/hapmap3r2B36/). TagSNPs for region chr4:84593218-84633217 were picked out for the CEU population using the Tagger multimarker algorithm with r² cutoff at 0.8 and minor allele frequency (MAF) cutoff at 0.05. MassARRAY was used for spectra acquisitions from the SpetroCHIP (Sequenom Inc.). Data analysis and genotype calling were done by using TyperAnalyzer Software version 4.0.3.18 (Sequenom Inc.). Each 384-well plate contained a minimum of eight nontemplate controls. For the c.1082G>A mutation, DNAs from three heterozygous mutation carriers were used as positive controls on each plate. For quality control, duplicate analysis was done for 6.5% of the samples from Oulu and for 6.7% of the samples from Kuopio.

### Statistical and Bioinformatical Analysis

Carrier frequencies between patients and healthy controls were compared by using Pearson Chi-Square or Fisher's exact test (two-sided, SPSS version 17.0 for Windows). All alterations were checked with NNSplice software for potential effects on splicing (http://www.fruitfiy.org/seq_tools/splice.html). Arg361Gln was tested for possible pathogenicity by using PolyPhen software (http://genetics.bwh.harvard.edu/pph). For tagSNP data, the overall association as well as the Hardy-Weinberg equilibrium, allele-specific P, odds ratio and confidence interval were computed with Cochran-Aimitage trend test.

### Tumor ER, PR, and HER2 Immunohistochemistry

Formalin-fixed, paraffin-embedded tissue sections (4 μm) were de-paraffinized and rehydrated in graded alcohols. Heat-induced epitope retrieval was performed with a digital pressure cooker in citrate buffer (pH 6.0) prior to application of the appropriate polyclonal (or monoclonal) antibody. The primary antibody was detected using the Envision+system (K4011, DakoCytomation) that employs horseradish peroxidase-labeled polymer conjugated to goat anti-rabbit immunoglobulin antibodies. The immune complexes were identified using a peroxidase reaction with 3,3′-diaminobenzidine-plus as chromogen. Slides were counterstained with Mayer's hematoxylin. Antibodies against ER (monoclonal, clone 1D5), PR (monoclonal, clone PgR636) and HER2 (polyclonal) were all purchased from Dako and used at 1:100, 1:200 and 1:1000, respectively.

### Antibodies Used in the Immunofluoresence and Immunoprecipitation Analysis for Functional Assessment of the Germline Abraxas R361Q Alteration

BRCA1 was detected by immunoblotting (TB) with mouse monoclonal antibody MS 110 at a 1:10 dilution, and by IF with a rabbit polyclonal antibody 07-434 (Millipore) at 1:500. A RAP80 rabbit polyclonal antibody was used for TB at 1:500 and IF at 1:100 dilutions. BRCC36 was detected by IB with a rabbit polyclonal antibody (12). MERIT40 was detected by TB at 1:1000 with a rabbit polyclonal antibody (14). Hemagglutinin (HA)-tagged proteins were detected by IB at 1:1000 and for IF at 1:1000 dilutions using mouse monoclonal antibody HA.11 (Covance).

### Cells

HeLa, 293T, MCF10A and U2OS cells were cultured in Dulbecco's modified Eagle's medium (DMEM) (Gibco) with 10% calf serum. Transient transfections were performed using LipoD293™ (SignaGen Laboratories) according to manufacturer's protocols.

### Cell Fractionation and Immunoprecipitation

293T cells were lysed in NETN-150 buffer [150 mM Tris-HCl (pH 7.4), 1 mM EDTA, 0.05% Nonidet P-40, 0.5 mM phenylmethylsulfonul fluoride, and 5 mM β-mercaptoethanol] for the production of whole cell extracts. Nuclear and cytoplasmic fractions were obtained from cells by Dounce homogenization of cells treated with hypotonic buffer [10 mM KCl, 10 mM Tris-HCl (pH 7.4), 1.5 mM MgCl₂]. Nuclei were lysed in NETN-150 buffer at 4° C. for 45 min. All subsequent processes were performed in NETN-150 buffer. The quality of the fractionation was evaluated by the detection of specific markers: γ-tubulin for the cytoplasmic fraction and proliferating cell nuclear antigen (PCNA) for the nuclear compartment.

### DNA Damage Induction

All radiation exposures were performed using a Gammacell 40 irradiator (Nordion International), which used cesium-137 as the radiation source.

Immunofluorescence Analysis

U2OS cells were cultured on glass coverslips and transiently transfected with LipoD293 (SignaGen Laboratories) according to manufacturer's protocols. Cells were treated with 10 Gy ionizing radiation and were allowed to recover for indicated times in a 37° C. incubator. Cells were washed with PBS and then fixed in 3% paraformaldehyde/2% sucrose containing solution for 10 min at room temperature. Cells were subsequently permeabilized with 0.5% triton solution for 5 min at 4° C. and then incubated with the appropriate primary antibody for 20 min at 37° C. Cells were then washed with PBST (PBS-Tween 20) and incubated with secondary antibody for 20 min at 37° C. Nuclei were then stained by incubating the cells in a PBS solution containing Hoechst 33258 dye (1 μg/ml). After four washes in PBST, coverslips were mounted onto glass slides using Vectashield mounting media (Vector Labs) and visualized using a Nikon Eclipse 80i fluorescent microscope.

To assess any potential activation and/or re-location of β-catenin, IF was performed with the mouse monoclonal f3-catenin antibody 610154 (BD Transduction Laboratories) and the rabbit polyclonal antibody 9110 (Abeam) directed against the HA tag. Both antibodies were used at the 1:1000 dilution. For IB, the mouse monoclonal antibody 12F7 directed against β-catenin was used at 1:1000.

### G₂ Checkpoint Assay

The G₂ checkpoint assay was performed by assessing the percentage of mitotic cells at 0 Gy and at 1 hr after 2 Gy IR as previously described (14). A rabbit polyclonal antibody against phosphorylated histone H3 (Upstate Biotechnology) was used to detect mitotic cells.

### Homology-Directed DNA Repair Assay

Homology-directed DNA repair assay was performed as described (34). U2OS cells containing stable integration of the direct repeat green fluorescent protein (DR-GFP) reporter locus were transiently transfected with I-Seel and Abraxas plasmids and then scored for homology-directed DNA repair by FACS for GFP-positive cells 72 hours after transfection.

### Plasmids

FLAG-HA-tagged version of Abraxas R361Q was created from addgene vector 27495.

### Results

A Genetic Screen for Abraxas Mutations Identified a R361Q Alteration that Associates with Familial Breast Cancer

125 affected index patients of Northern Finnish breast cancer families were screened for germline mutations in the Abraxas gene, revealing four intronic and six exonic variants (Table 3).

TABLE 3
Abraxas sequence variants observed in comprehensive mutation screening of familial breast cancer patients.

| | Nucleotide | Effect on | rs number | Frequency of heterozygotes, % (n/N) | | | | | P(OR; 95% | |
|---|---|---|---|---|---|---|---|---|---|---|
| Location | change | protein | or reference | FC | | USC | CT | | CI) | |
| Intron 2 | c.179−34_−38 | — | — | 0.8 | (1/125) | ND | 2.0 | (2/100) | 0.6 | (0.4; 0.4-4.4) |
| | delAATTA | | | | | | | | | |
| Intron 3 | c.216−44T>C | — | (21) | 2.4 | (3/125) | ND | 5.3 | (21/400) | 0.2 | (0.4; 0.1-1.5) |
| Intron 4 | c.282+46T>A | — | — | 0.8 | (1/125) | ND | — | (—/400) | 0.2 | (—) |
| Intron 7 | c.682−14A>G | — | — | 0.8 | (1/125) | ND | 0.5 | (2/400) | 0.6 | (1.6; 0.1-17.8) |
| Exon 9 | c.1042G>A | Ala348Thr | rs12642536 | 52.8 | (66/125) | ND | 51.1 | (92/180) | 0.8 | (1.1; 0.7-1.7) |
| | | | (20, 21) | | | | | | | |

| | | | | | | | | | | |
|---|---|---|---|---|---|---|---|---|---|---|
| Exon 9* | c.1082G>A | Arg361Gln | — | 2.4 | (3/125) | 0.1 | (1/991) | — | (—/868) | FC versus CT: |
| | | | | | | | | | | 0.002 (—) |
| | | | | | | | | | | FC versus |
| | | | | | | | | | | USC: 0.005 |
| | | | | | | | | | | (24.3; 2.5-235.9) |
| Exon 9 | c.1117G>A | Asp373Asn | rs13125836 | 12.8 | (16/125) | 16.5 | (163/990) | 13.9 | (121/868) | FC versus CT: |
| | | | (20, 21) | | | | | | | 0.8 (0.9; 0.5-1.6) |
| | | | | | | | | | | USC versus CT: |
| | | | | | | | | | | 0.1 (1.2; 0.9-1.6) |

| | | | | | | | | | | |
|---|---|---|---|---|---|---|---|---|---|---|
| Exon 9† | c.*249delG | — | rs34610900 | 53.6 | (67/125) | ND | 47.7 | (42/88) | 0.4 | (1.3; 0.7-2.2) |
| Exon 9† | c.*347C>T | — | rs60946531 | 0.8 | (1/125) | ND | — | (—/88) | 1.0 | (—) |
| | | | rs6825184 | | | | | | | |
| Exon 9† | c.*575A>G | — | — | 7.2 | (9/125) | ND | 5.7 | (5/88) | 0.8 | (1.3; 0.4-3.9) |

The following sequence information was used: ENSG00000163322 (genomic DNA, with the correction that rs60946531 is c.*347C>T), ENST00000321945 (mRNA), and ENSP00000369857 (protein). c.1082G>A and c.1117G>A: unselected cases (USC) and controls (CT) included patients from both Northern Finland and Northern Savo in Eastern Finland.
FC, familial cases;
OR, odds ratio;
CI, confidence interval;
ND, not determined.
*Carriers of c.1082G>A (in bold) also harbored the c.1042G>A and c.*249delG SNPs.
†3′ Untranslated region change.

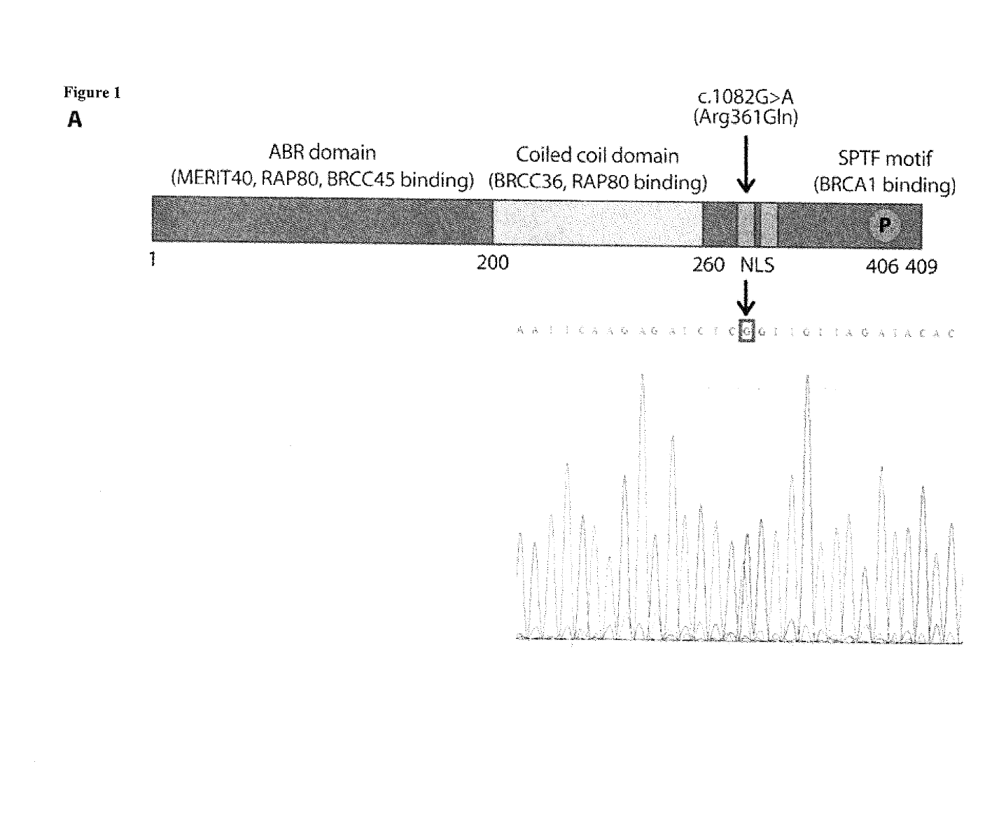

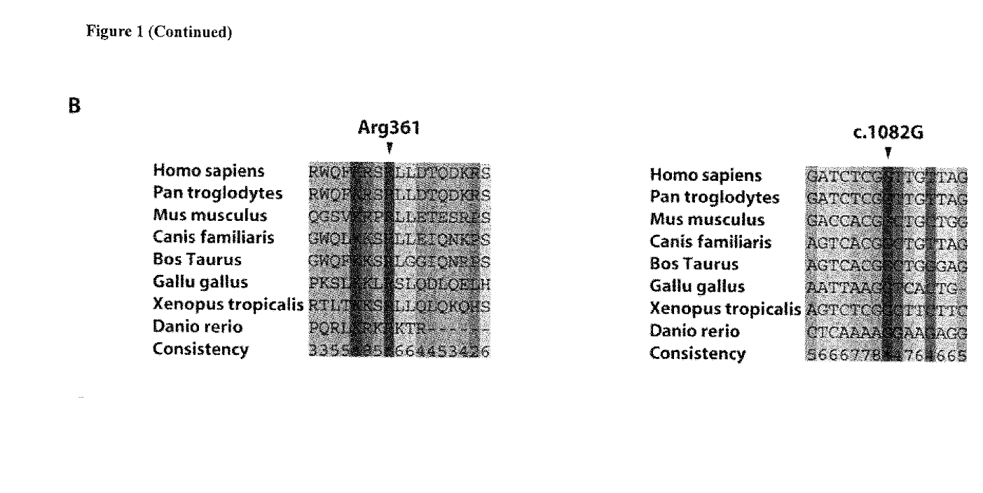

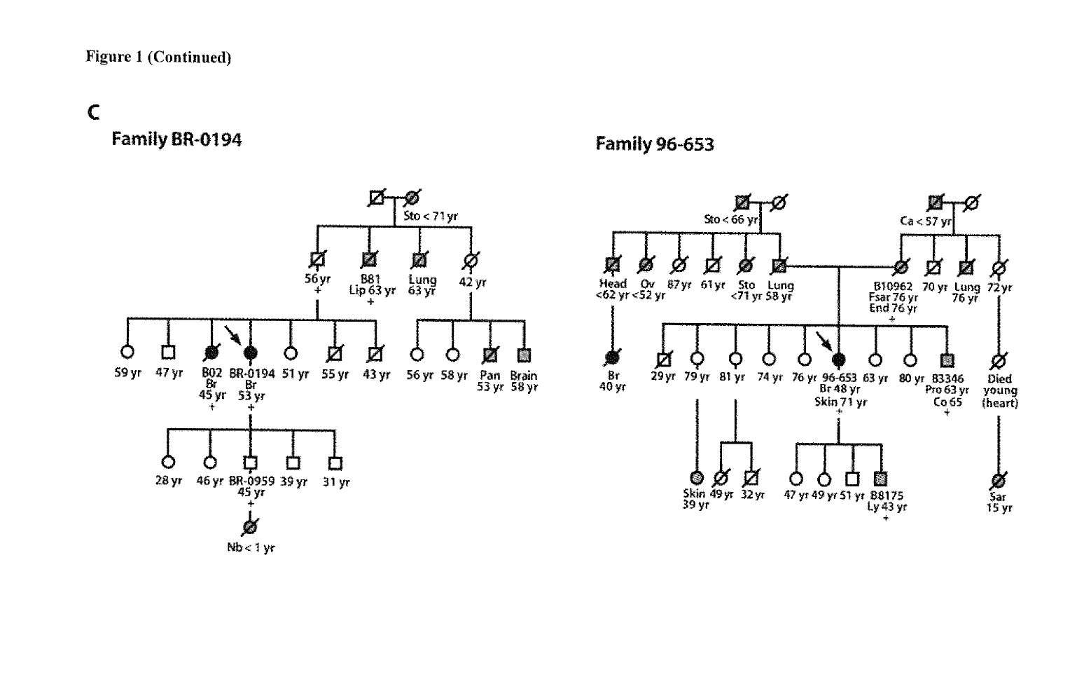

Altogether, five of the changes have not been reported either in the NCBI SNP database (http://www.ncbi.nlm.nih.gov/SNP/) or by previous studies (20, 21). A computer simulation analysis using PolyPhen software indicated that of the observed changes, only c.1082G>A was likely to result in functional changes in the protein. ESEfinder 2.0 and NNSplice software analysis did not reveal any likely abnormalities. Alteration c.1082G>A results in Arg361Gln (R361Q), which changes the last residue of a putative bipartite nuclear localization signal (NLS) (FIG. 1A) (10). Both Abraxas protein and mRNA sequence alignments revealed absolute evolutionary conservation among vertebrates at the site of the wildtype (WT) sequence (FIG. 1B). None of the studied common SNPs within Abraxas showed a statistically significant association with cancer susceptibility (Table 4).

TABLE 4
Association analysis of three Abraxas tagSNPs with breast cancer

| TagSNP | Minor allele | MAF cases | MAF controls | P-value^{a} |
|---|---|---|---|---|
| rs12499395 | T | 0.44 | 0.46 | 0.292 |
| rs12649417 | A | 0.50 | 0.48 | 0.179 |
| rs13125836 | A | 0.09 | 0.08 | 0.140 |

^{a}Cochran-Armitage trend test

Abraxas c.1082G>A alteration was observed in 3 out of 125 studied breast cancer families (2.4%), but was absent from 868 healthy female control individuals. The mutant allele was also identified in one out of 991 breast cancer cases unselected for a family history of the disease (Table 3). The prevalence of c.1082G>A in familial as compared with control cases, and also in familial compared with unselected breast cancer cases was found to be significantly different (P=0.002 and P=0.005, respectively), which indicates that this variant is disease-associated and specifically correlated with familial cancer. In agreement, the only mutation positive breast cancer patient in the unselected cohort also proved to have a familial cancer background (FIG. 1C, Family BR-0194).

Segregation analysis was performed in two of the mutation positive families (BR-0194 and 96-653, FIG. 1C), showing co-segregation between Abraxas c.1082G>A and cancer phenotype. Because of a lack of suitable DNA samples, segregation analysis was not possible for the two remaining Abraxas mutation positive breast cancer families (BR-02101 and 98-063). Of these, the index case of Family BR-02101 was diagnosed with both breast and endometrial cancer at the age of 37 and 49 years, respectively. She had a family history notable for stomach, lung, and prostate cancer, in addition to two maternal female cousins diagnosed with breast cancer at 48 and 54 years of age, respectively. The index case in the fourth family (98-063) was diagnosed with two ipsilateral primary breast tumors of different morphology (lobular and mucinous) at the age of 49 years. Her deceased sister displayed bilateral breast cancer at 53 and 71 years of age. The index patients of all four Abraxas mutation positive breast cancer families tested negative for mutations in BRCA1, BRCA2, TP53, CDH1 and PALB2. The average age of disease onset of the confirmed five Abraxas mutation positive breast cancer cases was 46 years (variation 35 to 53 years), similar to those with Finnish BRCA1 (46 years, variation 32 to 57 years) and BRCA2 (48 years, variation 45 to 67 years) mutations (22). For comparison, the mean age of onset for the recurrent Finnish PALB2 c.1592delT was found to be 52.9 years (variation 39 to 73 years), and the average age of onset for the unselected breast cancer cases was 57.9 years (variation 23-92) (23).

Morphology studies of R361Q positive breast cancers (Table 5) revealed a lobular phenotype in four out of the five tumors (80%). A second primary disease of mucinous morphology was observed in one of the patients that initially presented with lobular disease. This was in marked contrast to the single case (20%) of the ductal phenotype, which is typically seen in about 75% of breast cancer cases (24). Immunohistochemistry was performed on sections from the same breast cancer cases noted above, and all four of the informative tumors revealed strong expression of both the estrogen receptor (ER) and the progesterone receptor (PR), as well as total absence of human epidermal growth factor type 2 (HER2) expression (Table 5).

TABLE 5
Presentation of breast cancer, other tumors, and cellular dysplasia
in patients heterozygous for germline Abraxas c.1082G>A.

| | Breast cancer | | | | | |
|---|---|---|---|---|---|---|
| | | | | Age at diagnosis/ | | |
| Patient | Morphology | Receptor status* | TNM | metastasis (years) | Other cancer | Dysplasia |
| BR-0194 | Lobular | ER++, PR+++, | T2N1M0 | 53/bone, 62 | — | Endometrial |
| | | HER2− | | | | leiomyoma |
| B02 | Lobular | ER+++, PR+++, | T3- | 45/bone, 48; | — | — |
| | | HER2− | 4N1M0 | brain, 51 | | |
| BR-02101 | Ductal | ER+++, PR+++, | T2N0M0 | 35/— | Endometrial, | — |
| | | HER2− | | | 48 years | |
| 98-063 | Lobular/ | ER+++, PR+++, | T1 N0M0 | 49/— | | Colon tubular |
| | mucinous | HER2− | | | | adenoma (low- |
| | | | | | | grade dysplasia) |
| 96-653 | Lobular | NA | T1 N0M0 | 48/— | Skin, 71 | Colon tubular |
| | | | | | years (lentigo | adenoma (low- |
| | | | | | maligna) | grade dysplasia) |

TNM, tumor-nodemetastasis;
NA, data not available.
*Positive staining for the ER and PR is defined as nuclear immunostaining in 1 to 10% (+), 10 to 50% (++), or >50% (+++) of the tumor cells, whereas a minus (−) indicates negative staining.
Positive staining for HER2 is defined as membranous immunostaining of the tumor cells at levels + (faint positivity), ++ (moderate positivity), or +++ (strong, circumferential positivity), whereas HER2− indicates a completely negative staining.

These phenotypic observations suggest that Abraxas mutation-positive breast cancer cases can deviate from the pattern of hormone receptor and HER2 negativity associated with BRCA1 tumors (25). Tumors from patients with mutations in BRCA2 and PALB2, which, like Abraxas encode BRCA1-associated proteins, also frequently show hormone receptor positivity (23, 26).

BRCA1 and BRCA2 mutated cancers often display loss of heterozygosity (LOH), whereas loss of the wildtype allele at other BRCA associated breast cancer susceptibility genes is less common (27, 28). To test whether LOH had occurred in tumors of individuals heterozygous for the Abraxas mutation (a total of six R361Q-positive tumors), genomic DNA was extracted from pure or highly enriched tumor cell populations obtained by laser-capture microdissection from formalin-fixed, paraffin-embedded tumor tissue sections, and an Abraxas gene segment surrounding the c.1082G>A mutation was PCR-amplified and sequenced. The existence of the c.1082G>A mutation was confirmed in all studied tumors, whereas no evidence of loss of the wild-type allele was ever seen (Table 6).

TABLE 6
LOH analysis of laser-capture microdissected tumor cell DNA
of Abraxas c.1082G > A germline mutation carriers

| | | Confirmation of | |
|---|---|---|---|
| | | presence of germline | |
| | | Abraxas c.1082G > A | |
| | | mutation from patient's tumor | LOH status |
| Case | Tumor type | by sequencing analysis | for Abraxas |
| BR-0194 | Breast | Yes | Negative |
| B2 | Breast | Yes | Negative |
| BR-02101 | Endometrial | Yes | Negative |
| 98-063 | Breast | Yes | Negative |
| B10962 | Fibrosarcoma | Yes | Negative |
| B3346 | Colon | Yes | Negative |

### Abraxas R361Q Mutation Impairs Nuclear Localization and Disrupts BRCA1 DNA Damage Response Function

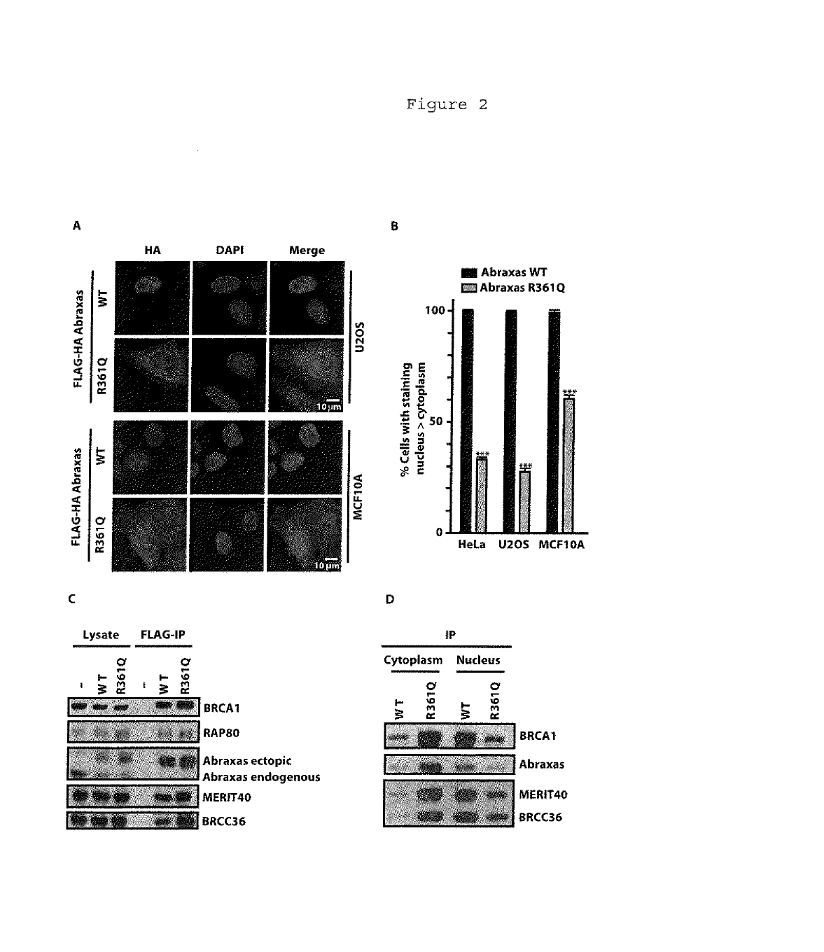

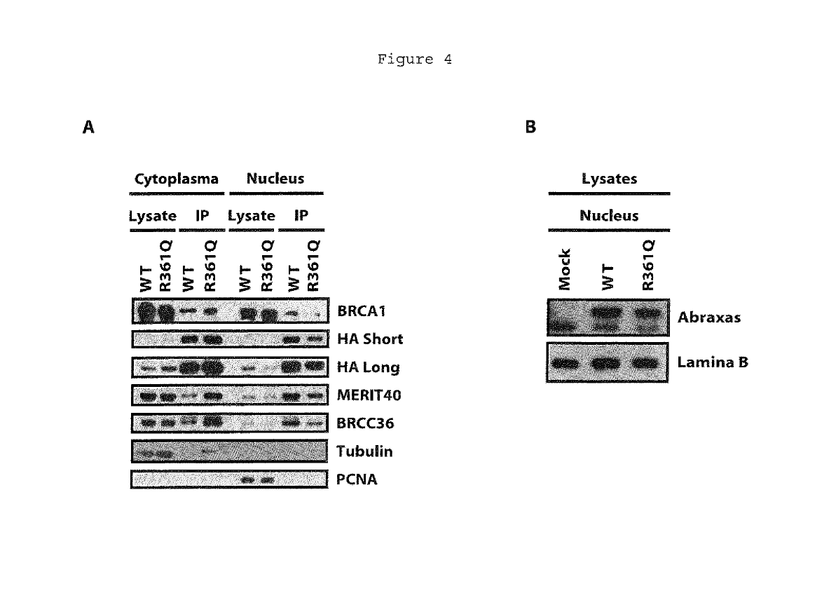

To assess the impact of R361Q on Abraxas subcellular localization, epitope tagged WT or R361Q mutant Abraxas were stably expressed in three different cell lines at near endogenous levels and subcellular localization examined by immunofluorescence (IF). WT Abraxas demonstrated predominantly nuclear localization by IF whereas Abraxas R361Q was primarily cytoplasmic (FIG. 2A, B). Coimmunoprecipitation experiments revealed that R361Q maintained interactions with BRCA1 and other core components of the holoezyme complex (FIG. 2C). Notably, Abraxas R361Q preferentially immunopreciptated with BRCA1 and other interacting partners in the cytoplasm, whereas wild-type Abraxas primarily displayed these interactions in the nucleus (FIG. 2D, and FIG. 4).

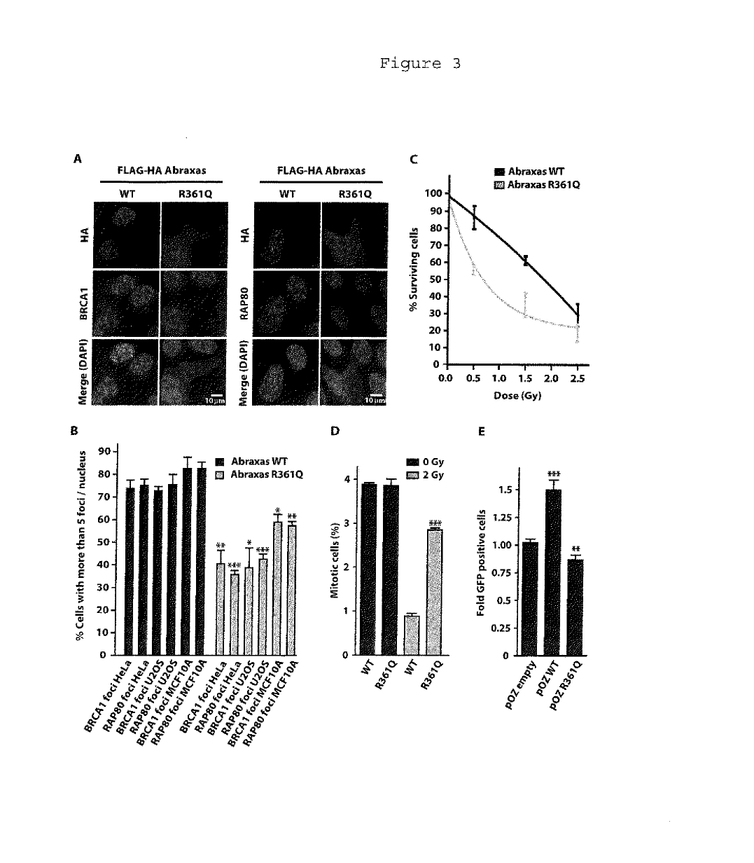

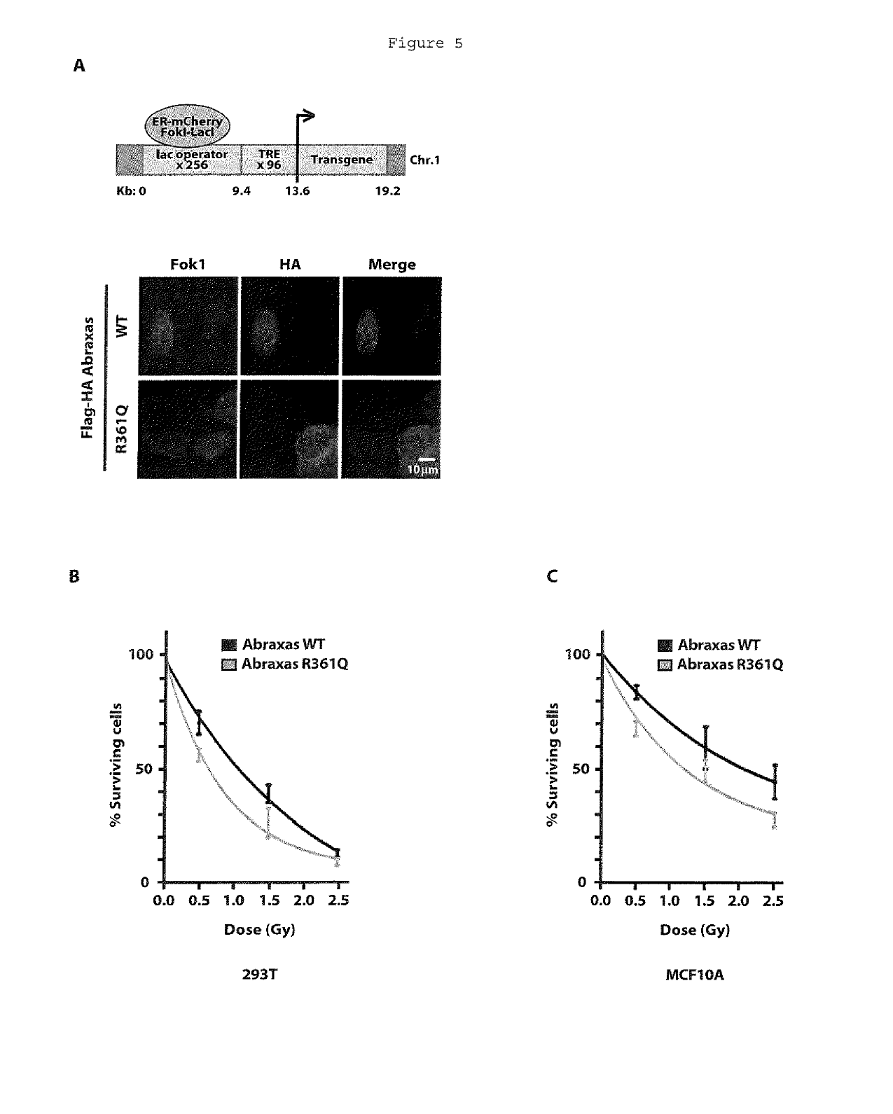

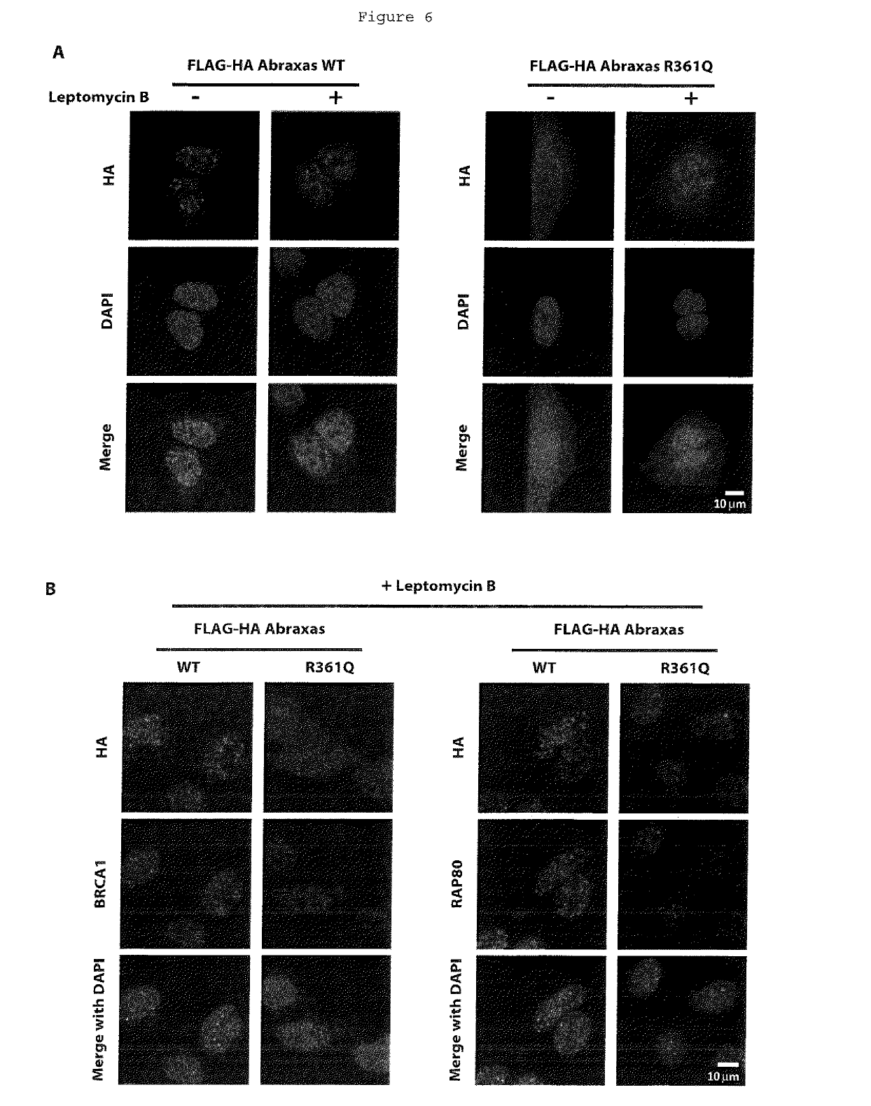

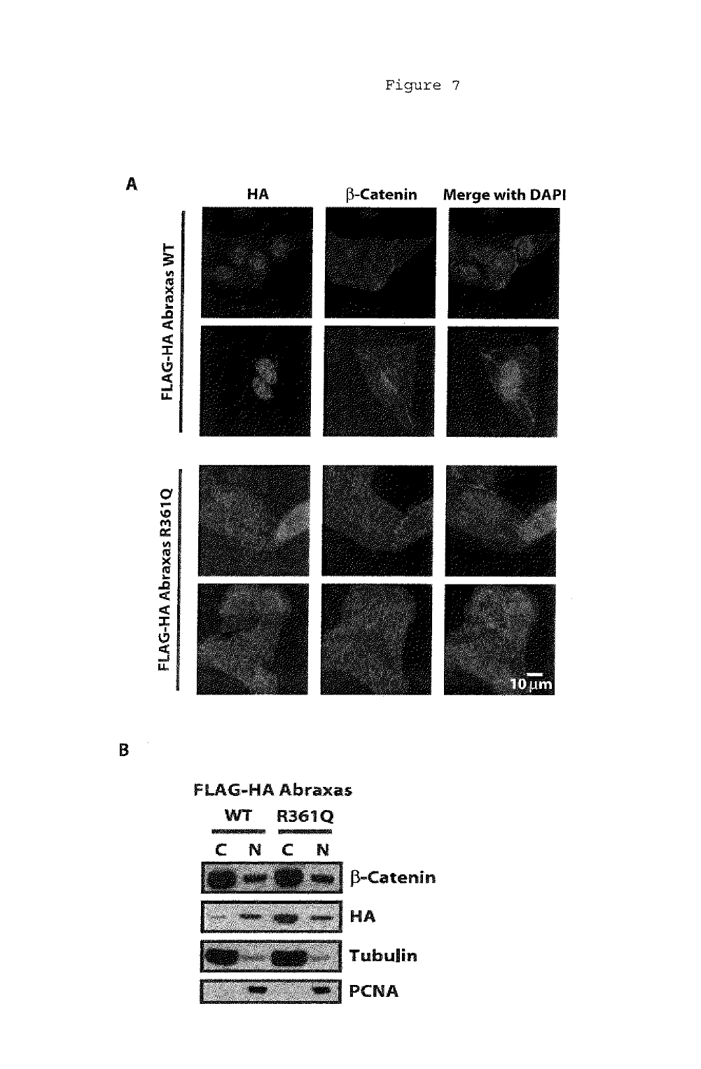

To determine the impact of R361Q on DNA damage response functions, recruitment of wild-type Abraxas or R361Q to DSBs was examined. R361Q recruitment to a specific site of nuclease induced DSBs (29) was strongly reduced in comparison to wild-type Abraxas, indicating deficiency in DNA damage response functions due to impaired nuclear accumulation (FIG. 5A). Similar results were observed at ionizing radiation induced foci (IRIF) in three different cell types (FIG. 3A, B). Consistent with a dominant-negative activity, R361Q expression significantly reduced both BRCA1 and RAP80 IRIF formation and resulted in IR hypersensitivity in three different cell lines (FIGS. 3A-C, and FIGS. 5C, D). In addition, R361Q expression partially disrupted the G2 checkpoint in response to IR and reduced the efficiency of homology directed DSB repair (FIG. 3D, E). These results indicate that reduced nuclear accumulation by Abraxas R361Q negatively impacts DSB localization of its interacting partners. In support of this concept, inhibition of nuclear export by leptomycin B treatment increased nuclear Abraxas R361Q and restored IRIF for Abraxas R361Q, BRCA1, and RAP80 (FIG. 6A, B). Conversely, Abraxas, R361Q expression did not aberrantly affect subcellular localization of β-catenin, further emphasizing the specificity for its impact on BRCA1 and RAP80 (FIG. 7). Abraxas, in addition to BRIP1 (FANCJ) and RAP80 (17, 27, 30), is now the third BRCA 1 BRCT-interacting partner with germline human breast cancer-associated mutations shown to exert dominant-negative effects on BRCA1 DNA repair function. These findings indicate that the nature of the Abraxas R361 Q mutation may obviate the need for LOH in tumors from affected patients.

### Discussion

Here, the identity of a novel, recurrent, constitutional mutation in the Abraxas gene in familial breast cancer is reported. Abraxas R361Q demonstrated exclusive association with cancer, segregation with disease within families, and loss of biological function in the DNA damage response. The impaired DNA damage response function extends beyond Abraxas itself, because Abraxas R361Q exerted a dominant-negative influence on BRCA1 and RAP80 by diminishing their accumulation at IR-induced DNA damage foci. In light of this finding, it is interesting that breast cancer associated mutation RAP80delE81 displayed similar dominant-negative properties with respect to BRCA1 localization to IR induced foci (17).

These observations complement existing knowledge of breast cancer-associated mutations within genes encoding proteins present in other BRCA1 containing protein complexes, reinforcing the concept of a BRCA-centered tumor suppressor network dedicated to the maintenance of genomic integrity (4, 31). Moreover, they establish ubiquitin recognition at DNA damage sites as a bona fide tumor suppression function of BRCA1-associated protein complexes. The BRCA1 protein complex containing Abraxas and RAP80 is unique in comparison to BRCA1 complexes that contain breast cancer suppressors PALB2, BRCA2, and BRIP1. RAP80 targets BRCA1 and Abraxas to ubiquitinated chromatin extending for a distance away from DSBs, whereas BRCA1 complexes containing BRCA2 and PALB2 or BRIP1 are thought to directly interact with DNA intermediates during homologous recombination-dependent DNA repair (32). Mutation in any of these genes results in severely reduced homologous recombination, whereas cells deficient for Abraxas or RAP80 display elevated use of homology-directed DNA repair mechanisms (33, 34). Abraxas R361 Q cells displayed slightly reduced homology-directed repair of a nuclease-induced DSB (FIG. 3E), indicating that Abraxas R361Q expression impairs BRCA1-dependent DNA repair differently than would an Abraxas-null allele. It is also unclear whether the IR hypersensitivity displayed in three different Abraxas R361Q-expressing cell lines was a result of the slight reduction in homology-directed DNA repair or alternatively due to defective G₂ checkpoint signaling (FIG. 3D). It is predicted that cancers expressing Abraxas or RAP80 dominant-negative mutant alleles would exhibit similar responses to chemotherapy regimens as tumors harboring mutations in other genes within the BRCA network. The Abraxas- or RAP80-deficient tumor models can be used to confirm such predictions.

In addition to breast cancer, Abraxas R361Q families displayed some relatively rare cancer types. Recently, a genome-wide association study associated a novel variant, rs1494961, located near Abraxas, with genetic susceptibility to upper aero-digestive tract cancers (35). In the present Example, both lung and lip cancer and lymphoma of the throat occurred in the two Abraxas c.1082G>A families shown in FIG. 1C. In addition, Family BR-0194 had a case of neuroblastoma; mutations in another BRCA1 associated gene, BARD1, has recently been connected to this disease (36). Therefore, Abraxas R361Q may also be associated with cancers other than breast cancer.

In conclusion, a coding variant of the Abraxas gene with a significantly different distribution in the familial cancer cases compared to the studied controls has been identified. This alteration is found in 2.4% of the studied Northern Finnish familial breast cancer cases and predominantly associates to a lobular tumor phenotype. Based on its exclusive occurrence in familial cancer cases, disease co-segregation, evolutionary conservation, and disruption of critical BRCA1 DNA damage response functions, the recurrent mutation connects to cancer predisposition. Similar to BRCA1 and BRCA2, mutations in Abraxas appear to be involved in susceptibility to certain other malignancy types beyond breast cancer.

### REFERENCES

1. Anglian Breast Cancer Study Group, Prevalence and penetrance of BRCA1 and BRCA2 mutations in a population-based series of breast cancer cases. Br. J. Cancer 83, 1301-1308 (2000).
2. D. F. Easton, K. A. Pooley, A. M. Dunning, P. D. Pharoah, D. Thompson, D. G. Ballinger, J. P. Struewing, J. Morrison, H. Field, R. Luben, N. Wareham, S. Ahmed, C. S. Healey, R. Bowman, SEARCH collaborators, K. B. Meyer, C. A. Haiman, L. K. Kolonel, B. E. Henderson, L. Le Marchand, P. Brennan, S. Sangrajrang, V. Gaborieau, F. Odefrey, C. Y. Shen, P. E. Wu, H. C. Wang, D. Eccles, D. G. Evans, J. Peto, O. Fletcher, N. Johnson, S. Seal, M. R. Stratton, N. Rahman, G. Chenevix-Trench, S. E. Bojesen, B. G. Nordestgaard, C. K. Axelsson, M. Garcia-Closas, L. Brinton, S. Chanock, J. Lissowska, B. Peplonska, H. Nevanlinna, R. Fagerholm, H. Eerola, D. Kang, K. Y. Yoo, D. Y. Noh, S. H. Ahn, D. J. Hunter, S. E. Hankinson, D. G. Cox, P. Hall, S. Wedren, J. Liu, Y. L. Low, N. Bogdanova, P. Schurmann, T. Dork, R. A. Tollenaar, C. E. Jacobi, P. Devilee, J. G. Klijn, A. J. Sigurdson, M. M. Doody, B. H. Alexander, J. Zhang, A. Cox, I. W. Brock, G. MacPherson, M. W. Reed, F. J. Couch, E. L. Goode, J. E. Olson, H. Meijers-Heijboer, A. van den Ouweland, A. Uitterlinden, F. Rivadeneira, R. L. Milne, G. Ribas, A. Gonzalez-Neira, J. Benitez, J. L. Hopper, M. McCredie, M. Southey, G. G. Giles, C. Schroen, C. Justenhoven, H. Brauch, U. Hamann, Y. D. Ko, A. B. Spurdle, J. Beesley, X. Chen, kConFab, AOCS Management Group, A. Manneiivaa, V. M. Kosma, V. Kataja, J. Hartikainen, N. E. Day, D. R. Cox, B. A. Ponder, Genome-wide association study identifies novel breast cancer susceptibility loci. Nature 447, 1087-1093 (2007).
3. M. R. Stratton, N. Rahman, The emerging landscape of breast cancer susceptibility. Nat. Genet. 40, 17-22 (2008).
4. T. Walsh, M. C. King, Ten genes for inherited breast cancer. Cancer Cell 11, 103-105 (2007).
5. A. Y. Shuen, W. D. Foulkes, Inherited mutations in breast cancer genes—risk and response. J. Mammary Gland Biol. Neoplasia 16, 3-15 (2011).
6. A. R. Venkitaraman, Cancer susceptibility and the functions of BRCA1 and BRCA2. Cell 108, 171-182 (2002).
7. X. Yu, C. C. Chini, M. He, G. Mer, J. Chen, The BRCT domain is a phospho-protein binding domain. Science 302, 639-642 (2003).
8. I. A. Manke, D. M. Lowery, A. Nguyen, M. B. Yaffe, BRCT repeats as phosphopeptide-binding modules involved in protein targeting. Science 302, 636-639 (2003).
9. B. Wang, S. Matsuoka, B. A. Ballif, D. Zhang, A. Smogorzewska, S. P. Gygi, S. J. Elledge, Abraxas and RAP80 form a BRCA1 protein complex required for the DNA damage response. Science 316, 1194-1198 (2007).
10. H. Kim, J. Huang, J. Chen, CCDC98 is a BRCA1-BRCT domain-binding protein involved in the DNA damage response. Nat. Struct. Mol. Biol. 14, 710-715 (2007).
11. Z. Liu, J. Wu, X. Yu, CCDC98 targets BRCA1 to DNA damage sites. Nat. Struct. Mol. Biol. 14, 716-720 (2007).
12. B. Sobhian, G. Shao, D. R. Lilli, A. C. Culhane, L. A. Moreau, B. Xia, D. M. Livingston, R. A. Greenberg, RAP80 targets BRCA1 to specific ubiquitin structures at DNA damage sites. Science 316, 1198-1202 (2007).
13. B. Wang, K. Hurov, K. Hofmann, S. J. Elledge, NBA1, a new player in the Brcal A complex, is required for DNA damage resistance and checkpoint control. Genes Dev. 23, 729-739 (2009).
14. G. Shao, J. Patterson-Fortin, T. E. Messick, D. Feng, N. Shanbhag, Y. Wang, R. A. Greenberg, MERIT40 controls BRCA1-Rap80 complex integrity and recruitment to DNA double-strand breaks. Genes Dev. 23, 740-754 (2009).
15. H. Kim, J. Chen, X. Yu, Ubiquitin-binding protein RAP80 mediates BRCA1-dependent DNA damage response. Science 316, 1202-1205 (2007).
16. L. Feng, J. Huang, J. Chen, MERIT40 facilitates BRCA1 localization and DNA damage repair. Genes Dev. 23, 719-728 (2009).
17. J. Nikkila, K. A. Coleman, D. Morrissey, K. Pylkäs, H. Erkko, T. E. Messick, S. M. Karppinen, A. Amelina, R. Winqvist, R. A. Greenberg, Familial breast cancer screening reveals an alteration in the RAP80 UIM domain that impairs DNA damage response function. Oncogene 28, 1843-1852 (2009).
18. A. C. Antoniou, X. Wang, Z. S. Fredericksen, L. McGuffog, R. Tarrell, O. M. Sinilnikova, S. Healey, J. Morrison, C. Kartsonaki, T. Lesnick, M. Ghoussaini, D. Barrowdale, EMBRACE, S. Peock, M. Cook, C. Oliver, D. Frost, D. Eccles, D. G. Evans, R. Eeles, L. Izatt, C. Chu, F. Douglas, J. Paterson, D. Stoppa-Lyonnet, C. Houdayer, S. Mazoyer, S. Giraud, C. Lasset, A. Remenieras, O. Caron, A. Hardouin, P. Berthet, GEMO Study Collaborators, F. B. Hogervorst, M. A. Rookus, A. Jager, A. van den Ouweland, N. Hoogerbrugge, R. B. van der Luijt, H. Meijers-Heijboer, E. B. Gomez Garcia, HEBON, P. Devilee, M. P. Vreeswijk, J. Lubinski, A. Jakubowska, J. Gronwald, T. Huzarski, T. Byrski, B. Gorski, C. Cybulski, A. B. Spurdle, H. Holland, kConFab, D. E. Goldgar, E. M. John, J. L. Hopper, M. Southey, S. S. Buys, M. B. Daly, M. B. Terry, R. K. Schmutzler, B. Wappenschmidt, C. Engel, A. Meindl, S. Preisler-Adams, N. Arnold, D. Niederacher, C. Sutter, S. M. Domchek, K. L. Nathanson, T. Rebbeck, J. L. Blum, M. Piedmonte, G. C. Rodriguez, K. Wakeley, J. F. Boggess, J. Basil, S. V. Blank, E. Friedman, B. Kaufman, Y. Laitman, R. Milgrom, I. L. Andrulis, G. Glendon, H. Ozcelik, T. Kirchhoff, J. Vijai, M. M. Gaudet, D. Altshuler, C. Guiducci, SWE-BRCA, N. Loman, K. Harbst, J. Rantala, H. Ehrencrona, A. M. Gerdes, M. Thomassen, L. Sunde, P. Peterlongo, S. Manoukian, B. Bonanni, A. Viel, P. Radice, T. Caldes, M. de la Hoya, C. F. Singer, A. Fink-Retter, M. H. Greene, P. L. Mai, J. T. Loud, L. Guidugli, N. M. Lindor, T. V. Hansen, F. C. Nielsen, I. Blanco, C. Lazard, J. Garber, S. J. Ramus, S. A. Gayther, C. Phelan, S. Narod, C. I. Szabo, MOD SQUAD, J. Benitez, A. Osorio, H. Nevanlinna, T. Heikkinen, M. A. Calige, M. S. Beattie, U. Hamann, A. K. Godwin, M. Montagna, C. Casella, S. L. Neuhausen, B. Y. Karlan, N. Tung, A. E. Toland, J. Weitzel, O. Olopade, J. Simard, P. Soucy, W. S. Rubinstein, A. Arason, G. Rennert, N. G. Martin, G. W. Montgomery, J. Chang-Claude, D. Flesch-Janys, H. Brauch, GENICA, G. Severi, L. Baglietto, A. Cox, S. S. Cross, P. Miron, S. M. Gerty, W. Tapper, D. Yannoukakos, G. Fountzilas, P. A. Fasching, M. W. Beckmann, I. Dos Santos Silva, J. Peto, D. Lambrechts, R. Paridaens, T. Rudiger, A. Forsti, R. Winqvist, K. Pylkäs, R. B. Diasio, A. M. Lee, J. Eckel-Passow, C. Vachon, F. Blows, K. Driver, A. Dunning, P. P. Pharoah, K. Offit, V. S. Pankratz, H. Hakonarson, G. Chenevix-Trench, D. F. Easton, F. J. Couch, A locus on 19p13 modifies risk of breast cancer in BRCA1 mutation carriers and is associated with hormone receptor-negative breast cancer in the general population. Nat. Genet. 42, 885-892 (2010).
19. K. L. Bolton, J. Tyrer, H. Song, S. J. Ramus, M. Notaridou, C. Jones, T. Sher, A. Gentry-Maharaj, E. Wozniak, Y. Y. Tsai, J. Weidhaas, D. Paik, D. J. Van Den Berg, D. O. Stram, C. L. Pearce, A. H. Wu, W. Brewster, H. Anton-Culver, A. Ziogas, S. A. Narod, D. A. Levine, S. B. Kaye, R. Brown, J. Paul, J. Flanagan, W. Sieh, V. McGuire, A. S. Whittemore, I. Campbell, M. E. Gore, J. Lissowska, H. P. Yang, K. Medrek, J. Gronwald, J. Lubinski, A. Jakubowska, N. D. Le, L. S. Cook, L. E. Kelemen, A. Brook-Wilson, L. F. Massuger, L. A. Kiemeney, K. K. Aben, A. M. van Altena, R. Houlston, I. Tomlinson, R. T. Palmieri, P. G. Moorman, J. Schildkraut, E. S. Iversen, C. Phelan, R. A. Vierkant, J. M. Cunningham, E. L. Goode, B. L. Fridley, S. Kruger-Kjaer, J. Blacker, E. Hogdall, C. Hogdall, J. Gross, B. Y. Karlan, R. B. Ness, R. P. Edwards, K. Odunsi, K. B. Moyisch, J. A. Baker, F. Modugno, T. Heikkinenen, R. Butzow, H. Nevanlinna, A. Leminen, N. Bogdanova, N. Antonenkova, T. Doerk, P. Hillemanns, M. Durst, I. Runnebaum, P. J. Thompson, M. E. Carney, M. T. Goodman, G. Lurie, S. Wang-Gohrke, R. Hein, J. Chang-Claude, M. A. Rossing, K. L. Cushing-Haugen, J. Doherty, C. Chen, T. Rafnar, S. Besenbacher, P. Sulem, K. Stefansson, M. J. Birrer, K. L. Terry, D. Hernandez, D. W. Cramer, I. Vergote, F. Arrant, D. Lambrechts, E. Despierre, P. A. Fasching, M. W. Beckmann, F. C. Thiel, A. B. Ekici, X. Chen, Australian Ovarian Cancer Study Group, Australian Cancer Study (Ovarian Cancer), Ovarian Cancer Association Consortium, S. E. Johnatty, P. M. Webb, J. Beesley, S. Chanock, M. Garcia-Closas, T. Sellers, D. F. Easton, A. Berchuck, G. Chenevix-Trench, P. D. Pharoah, S. A. Gayther, Common variants at 19p13 are associated with susceptibility to ovarian cancer. Nat. Genet. 42, 880-884 (2010).
20. A. Osorio, A. Barroso, M. J. Garcia, B. Martinez-Delgado, M. Urioste, J. Benitez, Evaluation of the BRCA1 interacting genes RAP80 and CCDC98 in familial breast cancer susceptibility. Breast Cancer Res. Treat. 113, 371-376 (2009).
21. D. J. Novak, N. Sabbaghian, P. Maillet, P. O. Chappuis, W. D. Foulkes, M. Tischkowitz, Analysis of the genes coding for the BRCA1-interacting proteins, RAP80 and Abraxas (CCDC98), in high-risk, non-BRCA1/2, multiethnic breast cancer cases. Breast Cancer Res. Treat. 117, 453-459 (2009).
22. L. Sarantaus, P. Huusko, H. Eerola, V. Launonen, P. Vehmanen, K. Rapakko, E. Gillanders, K. Syrjäkoski, T. Kainu, P. Vahteristo, R. Krahe, K. Pääckönen, J. Hartikainen, C. Blomqvist, T. Löppönen, K. Holli, M. Ryynanen, R. Butzow, A. Borg, B. Wasteson Arver, E. Holmberg, A. Mannermaa, J. Kere, O. P. Kallioniemi, R. Winqvist, H. Nevanlinna, Multiple founder effects and geographical clustering of BRCA1 and BRCA2 families in. Finland. Eur. J. Hum. Genet. 8, 757-763 (2000).
23. H. Erkko, B. Xia, J. Nikkila, J. Schleutker, K. Syrjäkoski, A. Mannermaa, A. Kallioniemi, K. Pylkäs, S. M. Karppinen, K. Rapakko, A. Miron, Q. Sheng, G. Li, H. Mattila, D. W. Bell, D. A. Haber, M. Grip, M. Reiman, A. Jukkola-Vuorinen, A. Mustonen, J. Kere, L. A. Aaltonen, V. M. Kosma, V. Kataja, Y. Soini, R. I. Drapkin, D. M. Livingston, R. Winqvist, A recurrent mutation in PALB2 in Finnish cancer families. Nature 446, 316-319 (2007).
24. C. I. Li, B. O. Anderson, J. R. Daling, R. E. Moe, Trends in incidence rates of invasive lobular and ductal breast carcinoma. JAMA 289, 1421-1424 (2003).
25. N. Mavaddat, A. C. Antoniou, D. F. Easton, M. Garcia-Closas, Genetic susceptibility to breast cancer. Mol. Oncol. 4, 174-191 (2010).
26. A. Borg, Molecular and pathological characterization of inherited breast cancer. Semin. Cancer Biol. 11, 375-385 (2001).
27. S. B. Cantor, D. W. Bell, S. Ganesan, E. M. Kass, R. Drapkin, S. Grossman, D. C. Wahrer, D. C. Sgroi, W. S. Lane, D. A. Haber, D. M. Livingston, BACH1, a novel helicase-like protein, interacts directly with BRCA1 and contributes to its DNA repair function. Cell 105, 149-160 (2001).
28. S. Casadei, B. M. Norquist, T. Walsh, S. Stray, J. B. Mandell, M. K. Lee, J. A. Stamatoyannopoulos, M. C. King, Contribution of inherited mutations in the BRCA2-interacting protein PALB2 to familial breast cancer. Cancer Res. 71, 2222-2229 (2011).
29. N. M. Shanbhag, I. U. Rafalska-Metcalf, C. Balane-Bolivar, S. M. Janicki, R. A. Greenberg, ATM-dependent chromatin changes silence transcription in cis to DNA double-strand breaks. Cell 141, 970-981 (2010).
30. S. Seal, D. Thompson, A. Renwick, A. Elliott, P. Kelly, R. Barfoot, T. Chagtai, H. Jayatilake, M. Ahmed, K. Spanova, B. North, L. McGuffog, D. G. Evans, D. Eccles, Breast Cancer Susceptibility Collaboration (UK), D. F. Easton, M. R. Stratton, N. Rahman, Truncating mutations in the Fanconi anemia J gene BRIP1 are low-penetrance breast cancer susceptibility alleles. Nat. Genet. 38, 1239-1241 (2006).
31. R. A. Greenberg, Recognition of DNA double strand breaks by the BRCA1 tumor suppressor network. Chromosoma 117, 305-317 (2008).
32. T. E. Messick, R. A. Greenberg, The ubiquitin landscape at DNA double-strand breaks. J. Cell Biol. 187, 319-326 (2009).
33. Y. Hu, R. Scully, B. Sobhian, A. Xie, E. Shestakova, D. M. Livingston, RAP80-directed tuning of BRCA1 homologous recombination function at ionizing radiation-induced nuclear foci. Genes Dev. 25, 685-700 (2011).
34. K. A. Coleman, R. A. Greenberg, The BRCA1-RAP80 complex regulates DNA repair mechanism utilization by restricting end resection. J. Biol. Chem. 286, 13669-13680 (2011).
35. J. D. McKay, T. Truong, V. Gaborieau, A. Chabrier, S. C. Chuang, G. Byrnes, D. Zaridze, O. Shangina, N. Szeszenia-Dabrowska, J. Lissowska, P. Rudnai, E. Fabianova, A. Bucur, V. Bencko, I. Holcatova, V. Janout, L. Foretova, P. Lagiou, D. Trichopoulos, S. Benhamou, C. Bouchardy, W. Ahrens, F. Merletti, L. Richiardi, R. Talamini, L. Barzan, K. Kjaerheim, G. J. Macfarlane, T. V. Macfarlane, L. Simonato, C. Canova, A. Agudo, X. Castellsague, R. Lowry, D. I. Conway, P. A. McKinney, C. M. Healy, M. E. Toner, A. Znaor, M. P. Curado, S. Koifinan, A. Menezes, V. Wunsch-Filho, J. E. Neto, L. F. Garrote, S. Boccia, G. Cadoni, D. Arzani, A. F. Olshan, M. C. Weissler, W. K. Funkhouser, J. Luo, J. Lubinski, J. Trubicka, M. Lener, D. Oszutowska, S. M. Schwartz, C. Chen, S. Fish, D. R. Doody, J. E. Muscat, P. Lazarus, C. J. Gallagher, S. C. Chang, Z. F. Zhang, Q. Wei, E. M. Sturgis, L. E. Wang, S. Franceschi, R. Herrero, K. T. Kelsey, M. D. McClean, C. J. Marsit, H. H. Nelson, M. Romkes, S. Buch, T. Nukui, S. Zhong, M. Lacko, J. J. Manni, W. H. Peters, R. J. Hung, J. McLaughlin, L. Vatten, I. Njolstad, G. E. Goodman, J. K. Field, T. Liloglou, P. Vineis, F. Clavel-Chapelon, D. Palli, R. Tumino, V. Krogh, S. Panico, C. A. Gonzalez, J. R. Quiros, C. Martinez, C. Navarro, E. Ardanaz, N. Larranaga, K. T. Khaw, T. Key, H. B. Bueno-de-Mesquita, P. H. Peeters, A. Trichopoulou, J. Linseisen, H. Boeing, G. Hallmans, K. Overvad, A. Tjonneland, M. Kumle, E. Riboli, K. Valk, T. Voodern, A. Metspalu, D. Zelenika, A. Boland, M. Delepine, M. Foglio, D. Lechner, H. Blanche, I. G. Gut, P. Galan, S. Heath, M. Hashibe, R. B. Hayes, P. Boffetta, M. Lathrop, P. Brennan, A genome-wide association study of upper aerodigestive tract cancers conducted within the INHANCE consortium. PLoS Genet. 7, e1001333 (2011).
36. M. Capasso, M. Devoto, C. Hou, S. Asgharzadeh, J. T. Glessner, E. F. Attiyeh, Y. P. Mosse, C. Kim, S. S. Diskin, K. A. Cole, K. Bosse, M. Diamond, M. Laudenslager, C. Winter, J. P. Bradfield, R. H. Scott, J. Jagannathan, M. Garris, C. McConville, W. B. London, R. C. Seeger, S. F. Grant, H. Li, N. Rahman, E. Rappaport, H. Hakonarson, J. M. Mans, Common variations in BARD 1 influence susceptibility to high-risk neuroblastoma. Nat. Genet. 41, 718-723 (2009).
37. J. M. Hartikainen, H. Tuhkanen, V. Kataja, M. Eskelinen, M. Uusitupa, V. M. Kosma, A. Mannermaa, Refinement of the 22q12-q13 breast cancer-associated region: evidence of TMPRSS6 as a candidate gene in an eastern Finnish population. Clin. Cancer Res. 12, 1454-1462 (2006).

The disclosed subject matter is not to be limited in scope by the specific embodiments described herein. Indeed, various modifications of the disclosed subject matter in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols are cited throughout this application, the disclosures of which are incorporated herein by reference in their entireties for all purpose.

What is claimed is:

1. A method of determining whether a subject has an increased risk for developing breast cancer comprising determining, in a biological sample comprising a nucleic acid comprising an Abraxas gene from the subject the nucleotide present at nucleotide position 1082 of the Abraxas gene, wherein presence of nucleotide other than G at nucleotide position 1082 is indicative of an increased risk of the subject developing breast cancer, as compared with a subject having a G at nucleotide position 1082.

2. The method of claim 1, wherein the presence of an A at nucleotide position 1082 is indicative of an increased risk of the subject developing breast cancer, as compared with a subject having a G at nucleotide position 1082.

3. The method of claim 1, wherein the Abraxas gene has the nucleotide sequence of SEQ ID NO: 1, or the complement thereof.

4. The method of claim 1, wherein said nucleic acid is a nucleic acid extract from a biological sample from said subject.

5. The method of claim 3, wherein said biological sample is blood or saliva.

6. The method of claim 1, wherein said testing comprises nucleic acid amplification.

7. The method of claim 7, wherein said nucleic acid amplification is carried out by polymerase chain reaction.

8. The method of claim 1, wherein said determining is performed using sequencing, 5′ nuclease digestion, molecular beacon assay, oligonucleotide ligation assay, size analysis, single-stranded conformation polymorphism analysis, or denaturing gradient gel electrophoresis (DGGE).

11. The method of claim 8, wherein said determining is performed using sequencing.

11. The method of claim 1, which is an automated method.

12. The method of claim 1, wherein said subject is heterozygous for said A allele.

13. The method of claim 1, wherein said breast cancer is lobular breast cancer.

14. The method of claim 1, wherein the subject has a family history of cancer.

15. The method of claim 15, wherein the cancer is breast cancer.

16. The method of claim 1, wherein the subject has previously tested negative for a BRCA1 or BRCA2 cancer-associated mutation.

17. The method of claim 1, wherein said method further comprises determining, in said biological sample, the sequence of one or more additional cancer susceptibility genes.

18. The method of claim 17, wherein said one or more additional cancer susceptibility genes include one or more additional breast cancer susceptibility genes.

19. A non-naturally-occurring nucleic acid molecule comprising all or a portion of the nucleic acid sequence of SEQ ID NO: 1 wherein said nucleic acid molecule is at least 10 nucleotides in length and wherein the nucleic acid sequence comprises a polymorphic site at nucleotide position 1082 of SEQ ID NO: 1.

20. The nucleic acid molecule according to claim 19, wherein the nucleotide at the polymorphic site is different from a nucleotide at the polymorphic site in a corresponding reference allele.


SEQUENCE LISTING (32 sequences)

SEQ ID NO: 1 - DNA, 1230 bp, Homo sapiens
atggagggggagagtacgtcggcggtgctctcgggctttgtgctcggcgcactcgctttccagcacctcaacacggactcggacacggaaggttttcttcttggggaagtaaaaggtgaagccaagaacagcattactgattcccaaatggatgatgttgaagttgtttatacaattgacattcagaaatatattccatgctatcagctttttagcttttataattcttcaggcgaagtaaatgagcaagcactgaagaaaatattatcaaatgtcaaaaagaatgtggtaggttggtacaaattccgtcgtcattcagatcagatcatgacgtttagagagaggctgcttcacaaaaacttgcaggagcatttttcaaaccaagaccttgtttttctgctattaacaccaagtataataacagaaagctgctctactcatcgactggaacattccttatataaacctcaaaaaggactttttcacagggtacctttagtggttgccaatctgggcatgtctgaacaactgggttataaaactgtatcaggttcctgtatgtccactggttttagccgagcagtacaaacacacagctctaaattttttgaagaagatggatccttaaaggaggtacataagataaatgaaatgtatgcttcattacaagaggaattaaagagtatatgcaaaaaagtggaagacagtgaacaagcagtagataaactagtaaaggatgtaaacagattaaaacgagaaattgagaaaaggagaggagcacagattcaggcagcaagagagaagaacatccaaaaagaccctcaggagaacatttttctttgtcaggcattacggaccttttttccaaattctgaatttcttcattcatgtgttatgtctttaaaaaatagacatgtttctaaaagtagctgtaactacaaccaccatctcgatgtagtagacaatctgaccttaatggtagaacacactgacattcctgaagctagtccagctagtacaccacaaatcattaagcataaagccttagacttagatgacagatggcaattcaagagatctcggttgttagatacacaagacaaacgatctaaagcagatactggtagtagtaaccaagataaagcatccaaaatgagcagcccagaaacagatgaagaaattgaaaagatgaagggttttggtgaatattcacggtctcctacattttga

SEQ ID NO: 2 - protein, 409 aa, Homo sapiens
MEGESTSAVLSGFVLGALAFQHLNTDSDTEGFLLGEVKGEAKNSITDSQMDDVEVVYTIDIQKYIPCYQLFSFYNSSGEVNEQALKKILSNVKKNVVGWYKFRRHSDQIMTFRERLLHKNLQEHFSNQDLVFLLLTPSIITESCSTHRLEHSLYKPQKGLFHRVPLVVANLGMSEQLGYKTVSGSCMSTGFSRAVQTHSSKFFEEDGSLKEVHKINEMYASLQEELKSICKKVEDSEQAVDKLVKDVNRLKREIEKRRGAQIQAAREKNIQKDPQENIFLCQALRTFFPNSEFLHSCVMSLKNRHVSKSSCNYNHHLDVVDNLTLMVEHTDIPEASPASTPQIIKHKALDLDDRWQFKRSRLLDTQDKRSKADTGSSNQDKASKMSSPETDEEIEKMKGFGEYSRSPTF

SEQ ID NO: 3 - DNA, 20 bp, Artificial sequence
  Synthetic oligonucleotide
gccctcgtcctcttgtgtag

SEQ ID NO: 4 - DNA, 19 bp, Artificial sequence
  Synthetic oligonucleotide
agggggagagaaggcagag

SEQ ID NO: 5 - DNA, 19 bp, Artificial sequence
  Synthetic oligonucleotide
ccccgactggtagcacata

SEQ ID NO: 6 - DNA, 20 bp, Artificial sequence
  Synthetic oligonuclotide
ttgcccagcctctgtatttc

SEQ ID NO: 7 - DNA, 20 bp, Artificial sequence
  Synthetic oligonucleotide
cttcctggcgtgaggtaaag

SEQ ID NO: 8 - DNA, 23 bp, Artificial sequence
  Synthetic oligonucleotide
cagagaaagaactgctagggttg

SEQ ID NO: 9 - DNA, 20 bp, Artificial sequence
  Synthetic oligonucleotide
gggctttggtagttgggtta

SEQ ID NO: 10 - DNA, 21 bp, Artificial sequence
  Synthetic oligonucleotide
gcaaccactttcaatttctgg

SEQ ID NO: 11 - DNA, 23 bp, Artificial sequence
  Synthetic oligonucleotide
aagaaagccattttaaggttgtt

SEQ ID NO: 12 - DNA, 24 bp, Artificial sequence
  Synthetic oligonucleotide
tgatgcgacaatatatgagaacac

SEQ ID NO: 13 - DNA, 23 bp, Artificial sequence
  Synthetic oligonucleotide
tttttaaatcttgtaggggacaa

SEQ ID NO: 14 - DNA, 23 bp, Artificial sequence
  Synthetic oligonucleotide
tcccttgaatttttattctgctg

SEQ ID NO: 15 - DNA, 21 bp, Artificial sequence
  Synthetic oligonucleotide
ggttttgtggttggtttttct

SEQ ID NO: 16 - DNA, 23 bp, Artificial sequence
  Synthetic oligonucleotide
catagccttcattaagcaactca

SEQ ID NO: 17 - DNA, 22 bp, Artificial sequence
  Synthetic oligonucleotide
tgcttgttactgacatcctcca

SEQ ID NO: 18 - DNA, 20 bp, Artificial sequence
  Synthetic oligonucleotide
cacctttgcactccaaccta

SEQ ID NO: 19 - DNA, 27 bp, Artificial sequence
  Synthetic oligonucleotide
ttgtcttagaatactgtggcatataaa

SEQ ID NO: 20 - DNA, 20 bp, Artificial sequence
  Synthetic oligonucleotide
tgagttccactggcctatcc

SEQ ID NO: 21 - DNA, 30 bp, Artificial sequence
  Synthetic oligonucleotide
acgttggatgcagctagtacaccacaaatc

SEQ ID NO: 22 - DNA, 30 bp, Artificial sequence
  Synthetic oligonucleotide
acgttggatggatcgtttgtcttgtgtatc

SEQ ID NO: 23 - DNA, 22 bp, Artificial sequence
  Synthetic oligonucleotide
tgtgtcttgtgtatctaacaac

SEQ ID NO: 24 - DNA, 30 bp, Artificial sequence
  Synthetic oligonucleotide
acgttggatgcatagttgacttaacagccc

SEQ ID NO: 25 - DNA, 30 bp, Artificial sequence
  Synthetic oligonucleotide
acgttggatgtcattggctaggaacagccc

SEQ ID NO: 26 - DNA, 17 bp, Artificial sequence
  Synthetic oligonucleotide
cccagctttggacctac

SEQ ID NO: 27 - DNA, 30 bp, Artificial sequence
  Synthetic oligonucleotide
acgttggatggacaagcatcatatggcacc

SEQ ID NO: 28 - DNA, 29 bp, Artificial sequence
  Synthetic oligonucleotide
acgttggatgcatcttctaaacgttctac

SEQ ID NO: 29 - DNA, 27 bp, Artificial sequence
  Synthetic oligonucleotide
ccctcttctaaacgttctacagataat

SEQ ID NO: 30 - DNA, 30 bp, Artificial sequence
  Synthetic oligonucleotide
acgttggatgggatgctttatcttggttac

SEQ ID NO: 31 - DNA, 30 bp, Artificial sequence
  Synthetic oligonucleotide
acgttggatgcaagagatctcggttgttag

SEQ ID NO: 32 - DNA, 20 bp, Artificial sequence
  Synthetic oligonucleotide
ttggttactactaccagtat